(19) United States
(12) Patent Application Publication
(10) Pub. No.: US 2024/0277774 A1
(43) Pub. Date: Aug. 22, 2024

(54) CELL COMPOSITIONS FOR TISSUE REGENERATION

(71) Applicant: BONUS THERAPEUTICS LTD., Haifa (IL)

(72) Inventors: Shai MERETZKI, Haifa (IL); Dror BEN-DAVID, Kiryat Motzkin (IL); Atara NOVIK, Haifa (IL); Ronit SHTRICHMAN, Haifa (IL)

(73) Assignee: BONUS THERAPEUTICS LTD., Haifa (IL)

(21) Appl. No.: 18/623,641

(22) Filed: Apr. 1, 2024

Related U.S. Application Data
(62) Division of application No. 16/316,730, filed on Jan. 10, 2019.
(60) Provisional application No. 62/360,500, filed on Jul. 11, 2016.

(51) Int. Cl. A61K 35/28; A61K 35/00; A61K 35/12; A61P 19/00; C12N 5/00; C12N 5/077; C12Q 1/68; C12Q 1/6876

(52) U.S. Cl. CPC A61K 35/28; A61K 35/12; A61P 19/00; C12N 5/0062; C12N 5/0653; C12Q 1/68; C12Q 1/6876; A61K 35/00; C12N 5/0075; C12N 5/0652; C12N 2501/155; C12Q 2600/158

(57) ABSTRACT

A composition comprising a cell population wherein the population is suitable for transplantation into a subject in need thereof, and characterized by differences of expression levels of a plurality of genes. Further, methods and kits for identifying a cell population suitable for transplantation into a subject in need thereof.

### CROSS REFERENCE TO RELATED APPLICATIONS

This application is a Divisional Application of patent application Ser. No. 16/316,730, filed on Jan. 10, 2019, which is a National Phase of PCT Patent Application No. PCT/IL2017/050789 having International filing date of Jul. 11, 2017, which claims the benefit of priority under 35 U.S.C. § 119(e) of U.S. Provisional Patent Application No. 62/360,500 titled “CELL COMPOSITIONS FOR TISSUE REGENERATION”, filed Jul. 11, 2016, the contents of which are incorporated herein by reference in their entirety.

### FIELD OF INVENTION

The present invention is generally in the field of tissue engineering, and particularly for use of cellular compositions for tissue regeneration and treatment of bone defects and disorders.

### BACKGROUND OF THE INVENTION

Tissue engineering and regenerative medicine provide exciting new treatments to help heal damaged organs and tissues. One important aspect of tissue engineering is the ability to use a person's own cells to treat that person. By using autologous cells, the risk of tissue rejection or graft rejection is eliminated.

One of the fastest growing segments of tissue engineering is in the treatment of bone disorders and disease. Bone has the ability to repair itself in response to injury. However, in complex clinical conditions, normal bone regeneration is impaired. These cases include large bone defects created by trauma, infection, tumor resection and skeletal abnormalities, or cases in which the regenerative process is compromised, including avascular necrosis and osteoporosis. Therapeutic approaches for bone repair include bone grafts substitutes and therapeutic molecules.

The bone regeneration market, and bone graft in particular is a growing market. The market growth is driven by several aspects, such as, increase in orthopedic procedures, increased aging population, increased preference for bone graft substitutes as replacements or complementary to autograft procedures, higher adoption of bone graft substitutes for orthopedic procedures and increase in reimbursement for orthopedic procedures.

Bone grafting is a surgical procedure that replaces missing bone. Bone grafting involves the use of either autologous grafts (i.e. using tissue from another part of the body of the patient), or of allografts (i.e. using tissue from a live human donor or cadaver). Therefore, a phase of tissue harvest from the patient or from a donor is required.

Tissue harvesting is typically executed by a surgical procedure usually involving collecting tissue from the iliac crest, the distal femur, the proximal tibia, the fibula, or from other small bones. The harvested tissue is restructured and transplanted at the damaged site.

However, the graft-harvesting procedures are associated with considerable morbidity and substantial pain. Tissue harvesting for an autologous grafts or from live donors for an allograft may also result in complications such as inflammation, infection, or even death.

The limited supply and inherited harvesting complications have inspired the development of alternative strategies for the repair of significant bone defects.

The use of 3-dimensional (3-D) bone substitutes such as bone extract, polymer or mineral scaffolds as implants has been investigated and porous biocompatible scaffolds have been used for the repair and regeneration of bone tissue.

Early attempts at tissue repair have focused mainly on the use of amorphous, biocompatible foam as porous plugs to fill large voids in bone. U.S. Pat. No. 4,186,448 described the use of porous mesh plugs composed of polyhydroxy acid polymers, such as polylactide, for healing bone voids. Several different methods for making other scaffolds were also described (e.g., U.S. Pat. Nos. 5,133,755; 5,514,378; 5,522,895; 5,607,474; 5,677,355; 5,686,091; 5,716,413; 5,755,792; 5,769,899; 5,770,193; 6,333,029; 6,365,149 and 6,534,084).

Bone marrow (BM) has been shown to contain population of cells that possess osteogenic potential. As such, an alternative to the scaffold-osteoinductive approach is to transplant into patients living cells that possess this capacity. Cytokine-manipulated, naïve autologous and allogeneic BM cells have successfully healed diffracted or resorbed bones in experimental models and human patients. Progenitor cells of the osteogenic lineage are seeded onto biocompatible (biodegradable or non-biodegradable) scaffolds in the presence or absence of growth promoting factors (e.g., U.S. Pat. Nos. 6,541,024; 6,544,290; 6,852,330). Transplantation into affected patients is performed following an ex-vivo expansion phase of the cells on the given scaffold. Using this approach, either primary osteogenic cells or expanded Mesenchymal Stromal Cells (MSC) layered upon ceramic scaffolds was able to regenerate bone tissue.

Living bone is a continuously evolving organ and in the normal course of bone maintenance, a constant remodeling process is being employed. In those procedures, old bone is being replaced by new bone and the organ responds to its environment changing requirements for strength and elasticity. Therefore, normal remodeling progression requires that the mechanical loading processes of bone resorption and bone formation procedures are tightly coordinated.

In cellular terms, this depends on sequential functioning of osteoclasts (bone resorbing cells) and osteoblasts (bone forming cells). In addition, endothelial cell and endothelial cell precursors (angioblasts) are required to form the new blood vessels in the developed bone tissue. Yet, the various cell types participating in bone formation are of different lineages. It is now known that osteoblasts stalk from mesenchymal stem cells, while osteoclasts (directly originating from Hematopoietic Stem Cells (HSC)) and endothelial cells are descendants of a common blast colony-forming cell. As such, methodologies for ex-vivo production of bone-like material that rely on osteoblasts as the exclusive cellular component suffer from an inherited fault.

### SUMMARY OF THE INVENTION

According to a first aspect the invention provides a composition comprising a cell population characterized by differences in expression levels of a plurality of genes, said plurality of genes is selected from at least two tables selected from tables 1-11, compared to control expression levels.

In some embodiments, the composition further comprises a mineral particle, wherein at least a portion of said cell population is in contact with (e.g., attached to) the mineral particle. In some embodiments, the mineral particle is selected from the group consisting of: coral mineral particle, cancellous bone and cortical bone.

According to another aspect, the invention provides a method for identifying a cell population, the method comprising determining the expression levels of a plurality of genes in a cell population, wherein differences in expression levels of a plurality of genes selected from the genes selected from at least two tables selected from tables 1-11, compared to a control expression levels, indicate identification of said cell population.

According to another aspect, the invention provides a method for identifying a cell population suitable for transplantation to a subject in need thereof, the method comprising determining the expression levels of a plurality of genes in a cell population, wherein differences in expression levels of a plurality of genes selected from the genes selected from at least two tables selected from tables 1-11, compared to a control expression levels, indicate that said cell population is suitable for transplantation.

In some embodiments, the plurality of genes is selected from one or more genes of each one of tables 1-11. In some embodiments, the plurality of genes comprises at least 50% of the genes listed in tables 1-11. In some embodiments, the plurality of genes is selected from genes listed in a table selected from tables 1-11.

In some embodiments, the cell population is derived from cells grown ex-vivo. In some embodiments, the cell population is derived from cells grown in a three dimensional culture. In some embodiments, the cell population is derived from human adipose tissue derived cells (HATDCs). In some embodiments, the cell population is derived from HATDCs subjected to osteogenic differentiation.

In some embodiments, the control expression levels correspond to a second cell population derived from cells grown in a two dimensional culture. In some embodiments, the second cell population is a cell population subjected to osteogenic differentiation.

In some embodiments, the osteogenic differentiation is induced by one or more osteogenic inducer selected from the group consisting of: bone morphogenic protein (BMP)-2, BMP-3, BMP-4, BMP-5, BMP-6 and BMP-7.

In some embodiments, the composition of the invention is for use in transplantation to a subject in need thereof.

In some embodiment, the differences in expression levels are, independently for each gene, selected from up-regulation, and down-regulation.

In some embodiments, the determining step of the method of the invention, comprises the step of obtaining nucleic acid molecules from said cell population. In some embodiments, the nucleic acids molecules are selected from mRNA molecules, DNA molecules and cDNA molecules. In some embodiments, the cDNA molecules are obtained by reverse transcribing said mRNA molecules.

In some embodiments, the determining step of the method of the invention, further comprises the step of hybridizing said nucleic acid molecules with a plurality of ligands each ligand capable of specifically complexing with, binding to, hybridizing to, or quantitatively detecting or identifying a single gene selected from the genes listed in Tables 1-11.

According to another aspect, the invention provides a kit comprising multiple ligands, each ligand capable of specifically complexing with, binding to, hybridizing to, or quantitatively detecting or identifying a single gene selected from a plurality of selected from at least two tables selected from tables 1-11. In some embodiments, the kit is for identifying a cell population suitable for transplantation to a subject. In some embodiments, the differences are selected from up-regulation, down-regulation, or a combination thereof. In some embodiments, the plurality of genes is selected from one or more genes of each one of tables 1-11. In some embodiments, the plurality of genes is selected from the genes listed in a table selected from tables 1-11. In some embodiments, the plurality of genes is selected from one or more genes of each one of tables 1-11. In some embodiments, the plurality of genes comprises at least 50% of the genes listed in tables 1-11.

In addition to the exemplary aspects and embodiments described above, further aspects and embodiments will become apparent by reference to the figures and by study of the following detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A-C are bar graphs showing qPCR analyses of: (A) BMP-2, (B) SP7, and (C) ALP, expressed in HADTCs cultured in 2D and 3D systems on mineral particles following 0, 1, 2, 3, or 4 days of osteogenic induction compared to untreated HADTCs cultured in 2D systems;

FIG. 2A-C are bar graphs showing qPCR analyses of: (A) BMP-2, (B) SP7, and (C) ALP, expressed in HADTCs cultured in 2D and 3D systems on mineral particles following 0, 1, 2, 3, or 4 days of osteogenic induction compared to untreated HADTCs cultured in 2D systems;

FIG. 3A-C are bar graphs showing qPCR analyses of: (A) BMP-2, (B) SP7, and (C) ALP, expressed in HADTCs cultured in 2D and 3D systems on mineral particles following 0, 1, 2, 3, or 4 days of osteogenic induction compared to untreated HADTCs cultured in 2D systems;

FIG. 4 is a bar graph analysis demonstrating the proportion of the variance component;

FIG. 5 is a Venn diagram demonstrating the number of differentially expressed genes (DEGs) resulting in each treatment group (A, B, or C) relative to control (BL);

FIG. 6A-C are graphs demonstrating the significance of differences in gene expressions for each treatment group (A) group A, (B) group B, and (C) group C, relative to control (BL), The y-axis of each graph represents the negative log 10 of the p-value, hence p value of 0.01 is represented by a value of 2 on the y axis, a p value of 0.001 is represented by a value of 3 on the y axis.

FIG. 7 is a Hierarchical Clustering (Heat map) for treatment groups A, B, C and BL.

FIG. 8 demonstrates a comparison analysis of differences in expression levels of genes in treatment groups A, B, and C relative to control (BL);

FIG. 9 demonstrates analysis of differences in expression levels of genes related to osteoblasts differentiation for treatment groups A, B and C, relative to control group (BL);

FIGS. 10A-B demonstrates analysis of differences in expression levels of genes related to angiogenesis and vascularization pathways for treatment groups (A) A and (B) B and C, relative to control group (BL);

FIG. 11 is a table (Table 11) listing exemplary differentially expressed genes (DEGs) of HADTCs cultured in 3D systems as compared to 2D systems.

### DETAILED DESCRIPTION OF THE INVENTION

The present invention, in some embodiments, provides a composition comprising a cell population characterized by a gene expression profile as shown in Tables 1-11. In some embodiments, the cell population is for transplantation, implantation, administration, and/or injection in a patient in need thereof. In some embodiments, the cell population is derived from cells grown ex-vivo.

In some embodiments, the invention provides a method for determining whether a composition is suitable for transplantation in a patient in need thereof. In additional embodiments, the invention provides a panel of genes useful for determining whether a composition is suitable for transplantation in a patient in need thereof.

The present invention is based, in part, on the finding that the cell population of the invention may be characterized by a gene expression signature of a plurality of genes. As exemplified herein below, expression levels of genes selected from Tables 1-11 may be used to distinguish between cells (e.g., Human Adipose Tissue Derived Cells or HATDCs) that were cultivated in 3-dimensional (3D) culture and/or subjected to osteogenic induction to other cells (e.g., HATDCs cultivated in 2-dimensional (2D) culture).

In some embodiments, the composition comprising the cell population as disclosed herein further comprises a mineral particle. In some embodiments, the composition is an implantable 3-dimensional (3D) composition useful for bone graft. In some embodiments, the cell population is derived from cells cultivated in 3D culture. In some embodiments, the cells cultivated in a 3D culture were further subjected to an osteogenic induction. In some embodiments, osteogenic differentiation is induced by osteogenic inducer (e.g., Bone Morphogenic Proteins (BMP)-2, BMP-3, BMP-4, BMP-5, B,P-6, or BMP-7). In some embodiments, the osteogenic differentiation is induced by BMP-2. In some embodiments, the osteogenic differentiation is induced by BMP-3. In some embodiments, the osteogenic differentiation is induced by BMP-4. In some embodiments, the osteogenic differentiation is induced by BMP-5. In some embodiments, the osteogenic differentiation is induced by BMP-6. In some embodiments, the osteogenic differentiation is induced by BMP-7.

In some embodiments, the cell population is derived HATDCs cultivated in 3D culture on a mineral scaffold and subjected to osteogenic induction.

In some embodiments, the cell population of the invention is a heterogeneous cell population. In some embodiments, the heterogeneous cell population allows various applications including adaptation to combined bone and cartilage graft for joint defects and/or bone vascularized graft. In some embodiments, the composition comprising the cell population is used for transplantation in a patient in need thereof. In another embodiment, the composition is used for filing a gap within a bone.

In some embodiments, the cell population of the invention has advantageous transplantation properties. In some embodiments, the cell population of the invention has improved transplant outcome. In some embodiments, said improved transplant outcome is a probability of more than 50%, more than 55%, more than 60%, more than 70%, more than 75%, more than 80%, more than 85%, more than 90%, more than 95%, more than 97%, more than 98%, or more than 99% of achieving successful transplantation (e.g., fusion of the transplanted cell population within said subject).

In some embodiments, the invention provides a method for determining whether a cell composition has a probability of more than 60%, more than 70%, more than 75%, more than 80%, more than 85%, more than 90%, or more than 95% of achieving successful transplantation (e.g., fusion of the transplanted cell population within said subject).

The term “subject” as used herein refers to an animal, e.g., a non-human mammals or a human. Non-human animal subjects may also include prenatal forms of animals, such as, e.g., embryos or fetuses. Non-limiting examples of non-human animals include: horse, cow, camel, goat, sheep, dog, cat, non-human primate, mouse, rat, rabbit, hamster, guinea pig and pig. In one embodiment, the subject is a human. Human subjects may also include fetuses. In one embodiment, a subject in need thereof is a subject afflicted with a fractured bone, a bone injury, diminished bone mass and/or bone abnormality.

As used herein, the terms implanting or implantation, transplanting or transplantation, administering or administration, injecting or injection, delivering or delivery, all refer to the process of providing a the composition disclosed herein to the site of treatment, and would be understood by a person of ordinary skill in the art to have the same meaning, depending on the composition properties and procedure employed for carrying out the delivery of tissue to the site. These terms can be used interchangeably and are in no way limiting to the method of the invention.

As used herein, the terms “gene expression profile”, “gene expression signature” or “gene expression fingerprint” are interchangeable, and refer to the pattern of gene expression modulation/difference, including increase or decrease of expression, exhibited by the heterogeneous cell population of the invention compared to populations derived from control cells (e.g. cells which were cultivated in a 2D culture and subjected or not subjected to osteogenic induction). The profile or fingerprint includes the relative degree of increase or decrease of expression of the “differentially expressed gene” (DEG) compared to control.

The terms “differentially expressed gene”, “DEG, “differential gene expression” and their synonyms, which are used interchangeably, refer to a gene whose expression is upregulated or downregulated to a higher or lower level in a selected population of cells compared to a control. It is also understood that a differentially expressed gene may be either activated or inhibited at the nucleic acid level or protein level, or may be subject to alternative splicing to result in a different polypeptide product. Such differences may be evidenced by a change in mRNA levels, surface expression, secretion or other partitioning of a polypeptide, for example.

As used herein, “difference in expression level”, and “modulation of expression level” and their synonyms, which are used interchangeably, refer to a significant difference in the expression of a gene. The terms encompasses increase in gene expression and/or decrease of gene expression.

The term “significant difference” in the context of the measured expression levels includes up-regulation/increase/induction and/or down-regulation/decrease/reduction, or combinations thereof of examined genes (such as that a first gene of the examined expression profile may be up-regulated whereas a second gene of the expression profile may be down-regulated).

In some embodiments, the determination of whether up-regulation or down-regulation of a specific gene indicates the tested population is suitable for transplantation is based on the data listed in Tables 1-11 (depicted using “+” or “−”). In some embodiments, said significant difference is a statistically significant difference such as in mean expression levels, as recognized by a skilled artisan. For example, without limitation, an increase or a decrease of about at least two folds, or alternatively of about at least three folds, compared to a control value is associated with a specific stage of differentiation of cells.

The terms “decrease”, “down-regulation” and “reduction” are used interchangeably herein to refer to a statistically significant decrease in gene expression. In some embodiments, decrease refers to at least 1.2, at least 1.3, at least 1.4, at least 1.5, at least 1.6, at least 1.7, at least 1.8, at least 1.9, at least 2, at least 3, at least 4, at least 5, at least 6, at least 7, at least 8, at least 9, or 10 folds decrease. Each possibility represents a separate embodiment of the present invention.

As used herein, the terms “increase”, “up-regulation” and “induction” are used interchangeably herein to refer to a statistically significant increase in gene expression. In some embodiments, increase refers to at least 1.2, at least 1.3, at least 1.4, at least 1.5, at least 1.6, at least 1.7, at least 1.8, at least 1.9, at least 2, at least 3, at least 4, at least 5, at least 6, at least 7, at least 8, at least 9, or 10 folds increase. Each possibility represents a separate embodiment of the present invention.

### The Composition

According to one aspect, there is provided a composition comprising a cell population, wherein the composition is characterized by a gene expression profile shown in any one of Tables 1-11. In some embodiments, the cell population is characterized by differences in expression levels of a plurality of genes selected from tables 1-10. In some embodiments, the cell population is characterized by differences in expression levels of a plurality of genes selected from tables 1-11. In some embodiments, the cell population is characterized by differences in expression levels of a plurality of genes selected from table 11.

In some embodiments, the differences in expression levels are determined compared to a control population. In some embodiment the control population is a population derived from cells cultivated in a 2 dimensional (2D) culture. In some embodiments, the control population is derived from cells cultivated in 2D culture and subjected to an osteogenic induction.

In some embodiments, the cell population is characterized by differences in expression levels of a plurality of genes selected from one or more genes selected from table 1, one or more genes selected from table 2, one or more genes selected from table 3, one or more genes selected from table 4, one or more genes selected from table 5, one or more genes selected from table 6, one or more genes selected from table 7, one or more genes selected from table 8, one or more genes selected from table 9, one or more genes selected from table 10, and/or one or more genes selected from table 11, or a combination thereof. In some embodiments, the plurality of genes comprises one or more genes from each one of tables 1-11. In some embodiments, the plurality of genes is selected from the genes listed in a Table selected from table 1-11. In some embodiments, one or more genes are at least two genes, or at least 3 genes, or at least 4 genes. Each possibility represents a separate embodiment of the instant invention.

In some embodiments, the cell population is characterized by differences in expression levels of a plurality of genes selected from table 1. In some embodiments, the cell population is characterized by differences in expression levels of a plurality of genes selected from table 2. In some embodiments, the cell population is characterized by differences in expression levels of a plurality of genes selected from table 3. In some embodiments, the cell population is characterized by differences in expression levels of a plurality of genes selected from table 4. In some embodiments, the cell population is characterized by differences in expression levels of a plurality of genes selected from table 5. In some embodiments, the cell population is characterized by differences in expression levels of a plurality of genes selected from table 6. In some embodiments, the cell population is characterized by differences in expression levels of a plurality of genes selected from table 7. In some embodiments, the cell population is characterized by differences in expression levels of a plurality of genes selected from table 8. In some embodiments, the cell population is characterized by differences in expression levels of a plurality of genes selected from table 9. In some embodiments, the cell population is characterized by differences in expression levels of a plurality of genes selected from table 10. In some embodiments, the cell population is characterized by differences in expression levels of a plurality of genes selected from table 11.

According to some embodiments, the plurality of genes comprises at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 55, at least 60, at least 65, at least 70, at least 75, at least 80, at least 85, at least 90, at least 95 different genes listed in Tables 1-11. Each possibility represents a separate embodiment of the instant invention.

According to some embodiments, the plurality of genes comprises at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most t 20, at most 21, at most 22, at most 23, at most 24, at most 25, at most 26, at most 27, at most 28, at most 29, at most 30, at most 31, at most 32, at most 33, at most 34, at most 35, at most 36, at most 37, at most 38, at most 39, at most 40, at most 41, at most 42, at most 43, at most 44, at most 45, at most 46, at most 47, at most 48, at most 49, at most 50, at most 55, at most 60, at most 65, at most 70, at most 75, at most 80, at most 85, at most 90, at most 95 different genes listed in Tables 1-11. Each possibility represents a separate embodiment of the instant invention.

According to some embodiments, the plurality of genes comprises at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most t 20, at most 21, at most 22, at most 23, at most 24, at most 25, at most 26, at most 27, at most 28, at most 29, at most 30, at most 31, at most 32, at most 33, at most 34, at most 35, at most 36, at most 37, at most 38, at most 39, at most 40, at most 41, at most 42, at most 43, at most 44, at most 45, at most 46, at most 47, at most 48, at most 49, at most 50, at most 55, at most 60, at most 65 genes listed in Table 11. Each possibility represents a separate embodiment of the instant invention.

According to some embodiments, the plurality of genes comprises at least 10%, at least 20%, at least 30%, at least 40%, at least 50% at least 60%, at least 70%, at least 80%, at least 90% of the genes listed in Tables 1-11. Each possibility represents a separate embodiment of the instant invention.

According to another embodiment, the plurality of genes comprises or consists of all the genes listed in Table 1. According to another embodiment, the plurality of genes comprises or consists of all the genes listed in Table 2. According to another embodiment, the plurality of genes comprises or consists of all the genes listed in Table 3. According to another embodiment, the plurality of genes comprises or consists of all the genes listed in Table 4. According to another embodiment, the plurality of genes comprises or consists of all the genes listed in Table 5. According to another embodiment, the plurality of genes comprises or consists of all the genes listed in Table 6. According to another embodiment, the plurality of genes comprises or consists of all the genes listed in Table 7. According to another embodiment, the plurality of genes comprises or consists of all the genes listed in Table 8. According to another embodiment, the plurality of genes comprises or consists of all the genes listed in Table 9. According to another embodiment, the plurality of genes comprises or consists of all the genes listed in Table 10. According to another embodiment, the plurality of genes comprises or consists of all the genes listed in Table 11.

### Gene Expression of the Cell Population of the Invention

### Down Regulation of MSCs Markers

As exemplified in the example section below, a cell population derived from cells cultivated in 3D culture is characterized by reduction of stem cell related genes, selected from: ANPEP (CD13), NT5E (CD73), THY1 (CD90), and KLF4 (as indicates in Table 1b).

In some embodiments, the cell population of the instant invention is characterized by differences in expression levels of one or more MSC marker genes listed in table 1, comprising: ANPEP (CD13), NT5E (CD73), THY1 (CD90), and KLF4. In some embodiments, the cell population of the instant invention is characterized by differences in expression levels of one or more genes selected from the group consisting of: ANPEP (CD13), NT5E (CD73), THY1 (CD90), and KLF4. In some embodiments, the cell population of the instant invention is characterized by decrease in expression levels of one or more genes selected from the group consisting of: ANPEP (CD13), NT5E (CD73), THY1 (CD90), and KLF4.

In some embodiments, decrease in expression levels of a one or more genes selected from the group consisting of: ANPEP (CD13), NT5E (CD73), THY1 (CD90), and KLF4, compared to a control population, indicates that the cell population is suitable for transplantation into a subject in need thereof. In some embodiments, decrease in expression level of NT5E (CD73) relative to a control population, indicates that the cell population is suitable for transplantation into a subject in need thereof.

TABLE 1
Gene expression of stem cells markers

| | | Expression |
|---|---|---|
| | Entrez Gene ID | relative to |
| Gene Name | (http://www.ncbi.nlm.nih.gov/gene) | control |
| ANPEP (CD13) | 290 | − |
| NT5E (CD73) | 4907 | − |
| THY1 (CD90) | 7070 | − |
| KLF4 | 9314 | − |

### Expression of Proliferation and Differentiation Regulatory Genes

As exemplified in the example section below, a cell population derived from cells cultivated in 3D culture is characterized by differences in gene expression levels as indicated in Table 2b. Further, a cell population derived from cells cultivated in 3D culture is characterized by decreased expression of proliferation regulatory genes selected from: AURKA, FOS, FGF2, BCL2L1, DDX21, RRAS2, STAT1, and ANXA2. Further, a cell population derived from cells cultivated in 3D culture is characterized by induction of expression levels of differentiation regulatory genes selected from: SFRP2, MRAS, NOX4, NOTCH3, and RGCC. As further exemplified in the example section, both HATDCs grown in 2D or 3D cultures that were subjected to osteogenic induction are characterized by differences in expression levels of proliferation regulator genes selected from the group consisting of: ID1, ID2, and ID3.

In some embodiments, the cell population of the instant invention is characterized by differences in expression levels of one or more genes listed in table 2, comprising: AURKA, FOS, FGF2, BCL2L1, DDX21, RRAS2, STAT1, ANXA2, SFRP2, MRAS, NOX4, NOTCH3, and RGCC. In some embodiments, a cell of the instant invention is characterized by differences in expression levels of one or more genes selected from the group consisting of: AURKA, FOS, FGF2, BCL2L1, DDX21, RRAS2, STAT1, ANXA2, SFRP2, MRAS, NOX4, NOTCH3, and RGCC, compared to control. In some embodiments, a cell of the instant invention is characterized by differences in expression levels of one or more genes selected from the group consisting of: AURKA, FGF2, BCL2L1, ANXA2, and SFRP2, compared to control.

In some embodiments, the cell population of the instant invention is characterized by decrease in expression levels of one or more genes selected from the group consisting of: AURKA, FOS, FGF2, BCL2L1, DDX21, RRAS2, STAT1, and ANXA2, compared to control. In some embodiments, a cell population of the instant invention is characterized by reduction of expression levels of one or more genes selected from the group consisting of: AURKA, FGF2, BCL2L1, and ANXA2, compared to control. In some embodiments, the cell population of the instant invention is characterized by increase in expression levels of one or more genes selected from the group consisting of: SFRP2, MRAS, NOX4, NOTCH3, and RGCC, compared to control. In some embodiments, the cell population of the instant invention is characterized by increase in an expression level of SFRP2, compared to control.

In some embodiments, differences in expression levels of one or more genes selected from the group consisting of: AURKA, FOS, FGF2, BCL2L1, DDX21, RRAS2, STAT1, ANXA2, SFRP2, MRAS, NOX4, NOTCH3, and RGCC, compared to a control population, indicates that the cell population is suitable for transplantation into a subject in need thereof. In some embodiments, induction of expression levels of one or more genes selected from the group consisting of: SFRP2, MRAS, NOX4, NOTCH3, and RGCC, compared to a control population, indicates that the cell population is suitable for transplantation into a subject in need thereof. In some embodiments, reduction of expression levels of one or more genes selected from the group consisting of: AURKA, FOS, FGF2, BCL2L1, DDX21, RRAS2, STAT1, and ANXA2, compared to a control population, indicates that the cell population is suitable for transplantation into a subject in need thereof. In some embodiments, induction of an expression level SFRP2, compared to a control population, indicates that the cell population is suitable for transplantation into a subject in need thereof.

TABLE 2
Gene expression of proliferation markers

| | | Expression |
|---|---|---|
| | Entrez Gene ID | relative |
| Gene Name | (http://www.ncbi.nlm.nih.gov/gene) | to control |
| AURKA | 6790 | − |
| FOS | 14281 | − |
| SFRP2 | 6423 | + |
| FGF2 (bFGF) | 2247 | − |
| BCL2L1 | 598 | − |
| MRAS | 22808 | + |
| NOX4 | 50507 | + |
| DDX21 | 9188 | − |
| RRAS2 | 22800 | − |
| STAT1 | 6772 | − |
| ANXA2 | 302 | − |
| NOTCH3 | 4854 | + |
| RGCC | 28984 | + |

### Expression of MHC I Genes

As exemplified in the example section below, a cell population derived from cells cultivated in 3D culture is characterized by induction of expression levels of MHCI genes compared to a control population cultured in 2D culture (as indicated in Table 3b).

As used herein, the term “MHC” refers to the Major Histocompatibility Complex, which involved in the presentation of foreign antigens to the immune system. As used herein, “HLA” is the human form of “MHC”. Typically, MHCI genes are expressed almost in all differentiated cells. Mesenchymal stem cells (MSCs) are known to express low levels of MHC class I molecules.

In some embodiments, the cell population of the instant invention is characterized by differences in expression levels of one or more genes listed in table 3, comprising: LA-A, HLA-B, HLA-DMA, HLA-F, HLA-G, and HLA-H. In some embodiments, the cell population of the instant invention is characterized by differences in expression levels of one or more genes compared to a control cell population. In some embodiments, the one or more genes are selected from the group consisting of: HLA-A, HLA-B, HLA-DMA, HLA-F, HLA-G, and HLA-H. In some embodiments, the one or more genes are selected from the group consisting of: HLA-A, HLA-B, HLA-F, HLA-G, and HLA-H. In some embodiments, differences in expression levels of one or more genes selected from the group consisting of: HLA-A, HLA-B, HLA-DMA, HLA-F, HLA-G, and HLA-H, indicate that the cell population is suitable for transplantation into a subject in need thereof. In some embodiments, increase in expression levels of a plurality of genes selected from the group consisting of: HLA-A, HLA-B, HLA-DMA, HLA-F, HLA-G, and HLA-H, compared to a control population, indicates that the cell population is suitable for transplantation into a subject in need thereof. In some embodiments, increase in expression levels of a plurality of genes selected from the group consisting of: HLA-A, HLA-B, HLA-F, HLA-G, and HLA-H, compared to a control population, indicates that the cell population is suitable for transplantation into a subject in need thereof.

TABLE 3
Gene expression of MHCI markers

| | | Expression |
|---|---|---|
| | | relative |
| Gene Name | Entrez Gene ID | to control |
| HLA-A | 3105 | + |
| HLA-B | 3106 | + |
| HLA-DMA | 3108 | + |
| HLA-F | 3133 | + |
| HLA-G | 3135 | + |
| HLA-H | 3136 | + |

### Expression of Adipocytes Markers

As exemplified in the example section below, a cell population derived from cells cultivated in 3D culture is characterized by differences in expression levels of a plurality of adipocyte markers genes compared to a control population cultured in 2D culture (as indicated in Table 4b).

In some embodiments, the cell population of the instant invention is characterized by differences in expression levels of one or more genes listed in table 4, comprising: PPARG, DLK1, ACSL1, AEBP1, and Sox9, compared to control. In some embodiments, the cell population of the instant invention is characterized by differences in expression levels of one or more genes selected from the group consisting of: PPARG, DLK1, ACSL1, AEBP1, and Sox9, compared to control. In some embodiments, the cell population of the instant invention is characterized by difference in an expression level of AEBP1.

In some embodiments, the cell population of the instant invention is characterized by reduction of expression levels of one or more genes selected from the group consisting of: PPARG, and ACSL1, compared to control. In some embodiments, the cell population of the instant invention is characterized by reduction of expression levels of PPARG, and ACSL1, compared to control. In some embodiments, the cell population of the instant invention is characterized by induction of expression levels of a plurality of adipocytes gene markers selected from the group consisting of: DLK1, AEBP1, and Sox9, compared to control. In some embodiments, the cell population of the instant invention is characterized by an induction of an expression level of AEBP1.

In some embodiments, differences in expression levels of one or more genes selected from the group consisting of: PPARG, DLK1, ACSL1, AEBP1, and Sox9, compared to a control population, indicates that the cell population is suitable for transplantation into a subject in need thereof. In some embodiments, induction of expression levels of one or more genes selected from the group consisting of: DLK1, AEBP1, and Sox9, compared to a control population, indicates that the cell population is suitable for transplantation into a subject in need thereof. In some embodiments, induction of expression level of AEBP1, compared to a control population, indicates that the cell population is suitable for transplantation into a subject in need thereof. In some embodiments, reduction of expression levels of a one or more genes selected from the group consisting of: PPARG, and ACSL1, compared to a control population, indicates that the cell population is suitable for transplantation into a subject in need thereof.

TABLE 4
Gene expression of adipocyte markers

| | | Expression |
|---|---|---|
| | Entrez Gene ID | relative |
| Gene Name | (http://www.ncbi.nlm.nih.gov/gene) | to control |
| PPARG | 5468 | − |
| DLK1 | 8788 | + |
| ACSL1 | 2180 | − |
| AEBP1 | 11568 | + |
| SOX9 | 6662 | + |

### Expression of Osteoblast Markers

As exemplified in the example section below, a cell population derived from cells cultivated in 3D culture is characterized by differences in expression levels of a plurality of osteoblasts markers genes compared to a control population cultured in 2D culture (as indicated in Table 5b).

In some embodiments, the cell population of the instant invention is characterized by differences in expression levels of a one or more osteoblast marker genes listed in table 5, comprising: BMP2, BMPR2, SP7, ALP (alkaline phosphatase), POSTN, FGFR3, Msx1 (Hox7), Msx2 (Hox8), DLX5, KAZALD1, CA12, BMPER, and FBN2. In some embodiments, the cell population of the instant invention is characterized by differences in expression levels of a one or more genes selected from the group consisting of: BMP2, BMPR2, SP7, ALP (alkaline phosphatase), POSTN, FGFR3, Msx1 (Hox7), Msx2 (Hox8), DLX5, KAZALD1, CA12, BMPER, and FBN2, compared to control. In some embodiments, the cell population of the instant invention is characterized by differences in expression levels of a one or more genes selected from the group consisting of: BMP2, ALP (alkaline phosphatase), POSTN, Msx1 (Hox7), Msx2 (Hox8), CA12, BMPER, and FBN2, compared to control

In some embodiments, the cell population of the instant invention is characterized by decrease in expression levels of one or more genes selected from the group consisting of: BMPER, and FBN2, compared to control. In some embodiments, the cell population of the instant invention is characterized by decrease in expression levels of BMPER, and FBN2, compared to control. In some embodiments, the cell population of the instant invention is characterized by a decrease in an expression level of FBN2, compared to control. In some embodiments, the cell population of the instant invention is characterized by induction of expression levels of one or more genes selected from the group consisting of: BMP2, BMPR2, SP7, ALP (alkaline phosphatase), POSTN, FGFR3, Msx1 (Hox7), Msx2 (Hox8), DLX5, KAZALD1, and CA12, compared to control. In some embodiments, the cell population of the instant invention is characterized by induction of expression levels of one or more genes selected from the group consisting of: BMP2, ALP, POSTIN, MSX1, MSX2, and CA12, compared to control. In some embodiments, the cell population of the instant invention is characterized by induction of expression levels of one or more genes selected from the group consisting of: BMP2, SP7, and ALP (alkaline phosphatase), compared to control.

In some embodiments, differences in expression levels of one or more genes selected from the group consisting of: BMP2, BMPR2, SP7, ALP (alkaline phosphatase), POSTN, FGFR3, Msx1 (Hox7), Msx2 (Hox8), DLX5, KAZALD1, CA12, BMPER, and FBN2, compared to a control population, indicate that the cell population is suitable for transplantation into a subject in need thereof. In some embodiments, differences in expression levels of a one or more genes selected from the group consisting of: BMP2, ALP (alkaline phosphatase), POSTN, Msx1 (Hox7), Msx2 (Hox8), CA12, and FBN2, compared to a control population, indicate that the cell population is suitable for transplantation into a subject in need thereof. In some embodiments, induction of expression levels of one or more genes selected from the group consisting of: BMP2, BMPR2, SP7, ALP (alkaline phosphatase), POSTN, FGFR3, Msx1 (Hox7), Msx2 (Hox8), DLX5, KAZALD1, CA12, compared to a control population, indicates that the cell population is suitable for transplantation into a subject in need thereof.

In some embodiments, induction of expression levels of one or more genes selected from the group consisting of: BMP2, ALP (alkaline phosphatase), POSTN, Msx1 (Hox7), Msx2 (Hox8), and CA12, compared to a control population, indicate that the cell population is suitable for transplantation into a subject in need thereof. In some embodiments, reduction of expression levels of a one or more genes selected from the group consisting of: BMPER, and FBN2, compared to a control population, indicates that the cell population is suitable for transplantation into a subject in need thereof. In some embodiments, reduction of expression level of FB2, compared to control population, indicates that the cell population is suitable for transplantation into a subject in need thereof.

TABLE 5
Gene expression of osteoblast markers

| | | Expression |
|---|---|---|
| | Entrez Gene ID | relative |
| Gene Name | (http://www.ncbi.nlm.nih.gov/gene) | to control |
| BMP2 | 650 | + |
| BMPR2 | 659 | + |
| SP7 | 121340 | + |
| ALP | 836158 | + |
| POSTN | 10631 | + |
| FGFR3 | 2261 | + |
| MSX1 (Hox7) | 4487 | + |
| MSX2 (Hox 8) | 4488 | + |
| DLX5 | 1749 | + |
| KAZALD1 | 81621 | + |
| CA12 | 771 | + |
| BMPER | 168667 | − |
| FBN2 | 2201 | − |

### Expression of Osteochondral Progenitors and Hypertrophic Chondrocytes Gene Markers

As exemplified in the example section below, a cell population derived from cells cultivated in 3D culture is characterized by differences in expression levels of a plurality of osteochondral progenitors and/or hypertrophic chondrocytes gene markers compared to a control population cultured in 2D culture (as indicated in Table 6b).

In some embodiments, the cell population of the instant invention is characterized by differences in expression levels of a one or more osteochondral progenitors and/or hypertrophic chondrocytes gene markers listed in table 6, comprising: Sox9, MGP, COL10A1, COL9A2, MMP13, GSN, CBFB, BAPX1 (NKX3-2), RUNX1, RUNX2, and COMP. In some embodiments, the cell population of the instant invention is characterized by differences in expression levels of one or more genes selected from the group consisting of: Sox9, MGP, COL10A1, COL9A2, MMP13, GSN, CBFB, BAPX1 (NKX3-2), RUNX1, RUNX2, and COMP, compared to control. In some embodiments, the cell population of the instant invention is characterized by differences in expression levels of one or more genes selected from the group consisting of: MMP13, RUNX1, and RUNX2, compared to control.

In some embodiments, the cell population of the instant invention is characterized by induction of expression levels of one or more genes selected from the group consisting of: Sox9, MGP, COL10A1, COL9A2, MMP13, GSN, CBFB, BAPX1 (NKX3-2), RUNX1, RUNX2, and COMP, compared to control. In some embodiments, the cell population of the instant invention is characterized by induction of expression levels of one or more genes selected from the group consisting of: MMP13, RUNX1, and RUNX2, compared to control.

In some embodiments, differences in expression levels one or more genes selected from the group consisting of: Sox9, MGP, COL10A1, COL9A2, MMP13, GSN, CBFB, BAPX1 (NKX3-2), RUNX1, RUNX2, and COMP, compared to a control population, indicate that the cell population is suitable for transplantation into a subject in need thereof. In some embodiments, induction of expression levels of one or more genes selected from the group consisting of: Sox9, MGP, COL10A1, COL9A2, MMP13, GSN, CBFB, BAPX1 (NKX3-2), RUNX1, RUNX2, and COMP, compared to a control population, indicates that the cell population is suitable for transplantation into a subject in need thereof. In some embodiments, induction of expression levels of one or more genes selected from the group consisting of: MMP13, RUNX1, and RUNX2, compared to a control population, indicates that the cell population is suitable for transplantation into a subject in need thereof.

TABLE 6
Gene expression of osteochondral progenitors and/or hypertrophic
chondrocytes gene markers

| | | Expression |
|---|---|---|
| | Entrez Gene ID | relative to |
| Gene Name | (http://www.ncbi.nlm.nih.gov/gene) | control |
| SOX9 | 6662 | + |
| MGP | 4256 | + |
| COL10A1 | 1300 | + |
| COL9A2 | 1298 | + |
| MMP13 | 4322 | + |
| GSN | 2934 | + |
| CBFB | 865 | + |
| BAPX1 (NKX3-2) | 579 | + |
| RUNX1 | 861 | + |
| RUNX2 | 860 | + |
| COMP | 1311 | + |

### Expression of ECM Gene Markers

As exemplified in the example section below, the cell population of the instant invention is characterized by differences in expression levels of a plurality of Extra cellular matrix (ECM) marker genes compared to a control population cultured in 2D culture (as indicated in Table 7b).

In some embodiments, the cell population of the instant invention is characterized by differences in expression levels of a one or more ECM marker genes listed in table 7, comprising: BGN, LAMA4, LAMA2, LTBP3, DPT, EFEMP2, PLOD1, TNC, DCN, FBLN2, NDNF, and SULF1. In some embodiments, the cell population of the instant invention is characterized by differences in expression levels of one or more genes selected from the group consisting of: BGN, LAMA4, LAMA2, LTBP3, DPT, EFEMP2, PLOD1, TNC, DCN, FBLN2, NDNF, and SULF1, compared to control. In some embodiments, the cell population of the instant invention is characterized by differences in expression levels of one or more genes selected from the group consisting of: BGN, LAMA4, LAMA2, DPT, PLOD1, DCN, and NDNF, compared to control.

In some embodiments, the cell population of the instant invention is characterized by induction of expression levels of one or more genes selected from the group consisting of: BGN, LAMA4, LAMA2, LTBP3, DPT, EFEMP2, PLOD1, TNC, DCN, FBLN2, NDNF, and SULF1, compared to control. In some embodiments, the cell population of the instant invention is characterized by induction of expression levels of one or more genes selected from the group consisting of: BGN, LAMA4, LAMA2, DPT, PLOD1, DCN, and NDNF, compared to control.

In some embodiments, differences in expression levels of one or more genes selected from the group consisting of: BGN, LAMA4, LAMA2, LTBP3, DPT, EFEMP2, PLOD1, TNC, DCN, FBLN2, NDNF, and SULF1, compared to a control population, indicate that the cell population is suitable for transplantation into a subject in need thereof. In some embodiments, induction of expression levels of one or more genes selected from the group consisting of: BGN, LAMA4, LAMA2, LTBP3, DPT, EFEMP2, PLOD1, TNC, DCN, FBLN2, NDNF, and SULF1, compared to a control population, indicates that the cell population is suitable for transplantation into a subject in need thereof. In some embodiments, induction of expression levels of one or more genes selected from the group consisting of: BGN, LAMA4, LAMA2, DPT, PLOD1, DCN, and NDNF, compared to a control population, indicates that the cell population is suitable for transplantation into a subject in need thereof.

TABLE 7
Gene expression of ECM gene markers

| | | Expression |
|---|---|---|
| | Entrez Gene ID | relative |
| Gene Name | (http://www.ncbi.nlm.nih.gov/gene) | to control |
| BGN | 633 | + |
| LAMA4 | 3910 | + |
| LAMA2 | 3908 | + |
| LTBP3 | 4054 | + |
| DPT | 1805 | + |
| EFEMP2 | 30008 | + |
| PLOD1 | 5351 | + |
| TNC | 3371 | + |
| DCN | 1634 | + |
| FBLN2 | 2199 | + |
| NDNF | 79625 | + |
| SULF1 | 23213 | + |

### Expression of Genes Encoding Structural Proteins

As exemplified in the example section below, a cell population derived from cells cultivated in 3D culture is characterized by differences in expression levels of one or more structural protein genes compared to a control population cultured in 2D culture (as indicated in Table 8b).

In some embodiments, the cell population of the instant invention is characterized by differences in expression levels of a one or more structural genes listed in table 8, comprising: MMP14, MMP2, MMP23B, MMP3, MMP7, COL16A1, COL24A1, COL6A2, COL7A1, COL8A2, ADAMTS2, and PCOLCE. In some embodiments, the cell population of the instant invention is characterized by differences in expression levels of one or more structural protein genes selected from the group consisting of: MMP14, MMP2, MMP23B, MMP3, MMP7, COL16A1, COL24A1, COL6A2, COL7A1, COL8A2, ADAMTS2, and PCOLCE, compared to control. In some embodiments, the cell population of the instant invention is characterized by differences in expression levels of one or more structural protein genes selected from the group consisting of: MMP14, MMP2, MMP23B, MMP3, COL6A2, COL7A1, COL8A2, and PCOLCE, compared to control.

In some embodiments, the cell population of the instant invention is characterized by induction of expression levels of one or more structural protein genes selected from the group consisting of: MMP14, MMP2, MMP23B, MMP3, MMP7, COL16A1, COL24A1, COL6A2, COL7A1, COL8A2, ADAMTS2, and PCOLCE, compared to control. In some embodiments, the cell population of the instant invention is characterized by induction of expression levels of one or more structural protein genes selected from the group consisting of: MMP14, MMP2, MMP23B, MMP3, COL6A2, COL7A1, COL8A2, and PCOLCE, compared to control.

In some embodiments, differences in expression levels of one or more structural protein genes selected from the group consisting of: MMP14, MMP2, MMP23B, MMP3, MMP7, COL16A1, COL24A1, COL6A2, COL7A1, COL8A2, ADAMTS2, and PCOLCE, compared to a control population, indicate that the cell population is suitable for transplantation into a subject in need thereof. In some embodiments, induction of expression levels of one or more structural protein genes selected from the group consisting of MMP14, MMP2, MMP23B, MMP3, MMP7, COL16A1, COL24A1, COL6A2, COL7A1, COL8A2, ADAMTS2, and PCOLCE, compared to a control population, indicates that the cell population is suitable for transplantation into a subject in need thereof. In some embodiments, induction of expression levels of one or more structural protein genes selected from the group consisting of: MMP14, MMP2, MMP23B, MMP3, COL6A2, COL7A1, COL8A2, and PCOLCE, compared to a control population, indicates that the cell population is suitable for transplantation into a subject in need thereof.

TABLE 8
Gene expression of structural proteins

| | | Expression |
|---|---|---|
| | Entrez Gene ID | relative |
| Gene Name | (http://www.ncbi.nlm.nih.gov/gene) | to control |
| MMP14 | 4323 | + |
| MMP2 | 4313 | + |
| MMP23B | 8510 | + |
| MMP3 | 4314 | + |
| MMP7 | 4316 | + |
| COL16A1 | 1307 | + |
| COL24A1 | 255631 | + |
| COL6A2 | 1292 | + |
| COL7A1 | 1294 | + |
| COL8A2 | 1296 | + |
| ADAMTS2 | 9509 | + |
| PCOLCE | 5118 | + |

### Expression of Angiogenic and Vasculogenic Related Genes

As exemplified in the example section below, a cell population derived from cells cultivated in 3D culture is characterized by differences in expression levels of one or more vascular related marker genes compared to a control population cultured in 2D culture (as indicated in Table 9b).

In some embodiments, the cell population of the instant invention is characterized by differences in expression levels of a one or more angiogenic and vasculogenic related genes listed in table 9, comprising: TBX2, TBX3, ANG, ANGPT2, ANGPTL2, TRO, EDNRA, EPHA2, F2R, PGF, CTHRC1, PTGDS, AEBP1, IL8 (Cxcl8), IL11, HEY1, ECM1, MFGE8, and SRPX2, and UNC5B. In some embodiments, the cell population of the instant invention is characterized by differences in expression levels of one or more genes selected from the group consisting of: TBX2, TBX3, ANG, ANGPT2, ANGPTL2, TRO, EDNRA, EPHA2, F2R, PGF, CTHRC1, PTGDS, AEBP1, IL8 (Cxcl8), IL11, HEY1, ECM1, MFGE8, and SRPX2, and UNC5B, compared to control. In some embodiments, the cell population of the instant invention is characterized by differences in expression levels of one or more genes selected from the group consisting of: ANGPT2, ANGPTL2, TRO, PTGDS, AEBP1, IL8 (Cxcl8), and ECM1, compared to control.

In some embodiments, the cell population of the instant invention is characterized by induction of expression levels of one or more genes selected from the group consisting of: TBX2, TBX3, ANG, ANGPT2, ANGPTL2, TRO, EDNRA, EPHA2, F2R, PGF, CTHRC1, PTGDS, AEBP1, IL8 (Cxcl8), IL11, HEY1, ECM1, MFGE8, and SRPX2, and UNC5B, compared to control. In some embodiments, the cell population of the instant invention is characterized by induction of expression levels of one or more genes selected from the group consisting of: ANGPT2, ANGPTL2, TRO, PTGDS, AEBP1, IL8 (Cxcl8), and ECM1, compared to control.

In some embodiments, differences in expression levels of one or more genes selected from the group consisting of: TBX2, TBX3, ANG, ANGPT2, ANGPTL2, TRO, EDNRA, EPHA2, F2R, PGF, CTHRC1, PTGDS, AEBP1, IL8 (Cxcl8), IL11, HEY1, ECM1, MFGE8, and SRPX2, and UNC5B, compared to a control population, indicate that the cell population is suitable for transplantation into a subject in need thereof. In some embodiments, induction of expression levels of one or more genes selected from the group consisting of: TBX2, TBX3, ANG, ANGPT2, ANGPTL2, TRO, EDNRA, EPHA2, F2R, PGF, CTHRC1, PTGDS, AEBP1, IL8 (Cxcl8), IL11, HEY1, ECM1, MFGE8, and SRPX2, and UNC5B, compared to a control population, indicates that the cell population is suitable for transplantation into a subject in need thereof. In some embodiments, induction of expression levels of one or more genes selected from the group consisting of: ANGPT2, ANGPTL2, TRO, PTGDS, AEBP1, IL8 (Cxcl8), and ECM1, compared to a control population, indicates that the cell population is suitable for transplantation into a subject in need thereof.

TABLE 9
Gene expression of angiogenic and
vasculogenic related genes

| | | Expression |
|---|---|---|
| | Entrez | relative |
| Gene Name | Gene ID | to control |
| TBX2 | 6909 | + |
| TBX3 | 6926 | + |
| ANG | 283 | + |
| ANGPT2 | 285 | + |
| ANGPTL2 | 23452 | + |
| TRO | 7216 | + |
| EDNRA | 1909 | + |
| EPHA2 | 1969 | + |
| F2R | 2149 | + |
| PGF | 5228 | + |
| CTHRC1 | 115908 | + |
| PTGDS | 5730 | + |
| AEBP1 | 165 | + |
| IL8 (Cxcl8) | 3576 | + |
| IL11 | 3598 | + |
| HEY1 | 23462 | + |
| ECM1 | 1893 | + |
| MFGE8 | 4240 | + |
| SRPX2 | 27286 | + |
| UNC5B | 219699 | + |

### Expression of Specific Upstream Regulators

As exemplified in the example section below, a cell population derived from cells cultivated in 3D culture is characterized by differences in expression levels of one or more upstream regulator genes compared to a control population cultured in 2D culture (as indicated in Table 10b).

In some embodiments, the cell population of the instant invention is characterized by differences in expression levels of a one or more upstream regulator genes listed in table 10, comprising: TGFB3, BAMB1, IGFBP2, and IGFBP5. In some embodiments, the cell population of the instant invention is characterized by differences in expression levels of one or more upstream regulator genes selected from the group consisting of: TGFB3, BAMB1, IGFBP2, and IGFBP5, compared to control. In some embodiments, the cell population of the instant invention is characterized by differences in expression levels of one or more upstream regulator genes selected from the group consisting of: TGFB3, IGFBP2, and IGFBP5, compared to control.

In some embodiments, the cell population of the instant invention is characterized by induction of expression levels of one or more upstream regulator genes selected from the group consisting of: TGFB3, BAMB1, IGFBP2, and IGFBP5, compared to control. In some embodiments, the cell population of the instant invention is characterized by induction of expression levels of one or more upstream regulator genes selected from the group consisting of: TGFB3, IGFBP2, and IGFBP5, compared to control.

In some embodiments, differences in expression levels of one or more upstream regulator genes selected from the group consisting of: TGFB3, BAMB1, IGFBP2, and IGFBP5, compared to a control population, indicate that the cell population is suitable for transplantation into a subject in need thereof. In some embodiments, induction of expression levels of one or more upstream regulator genes selected from the group consisting of: TGFB3, BAMB1, IGFBP2, and IGFBP5, compared to a control population, indicates that the cell population is suitable for transplantation into a subject in need thereof. In some embodiments, induction of expression levels of one or more upstream regulator genes selected from the group consisting of: TGFB3, IGFBP2, and IGFBP5, compared to a control population, indicates that the cell population is suitable for transplantation into a subject in need thereof.

TABLE 10
Gene expression of upstream regulators

| | | Expression |
|---|---|---|
| | Entrez | relative |
| Gene Name | Gene ID | to control |
| TGFB3 | 7043 | + |
| BAMBI | 25805 | + |
| IGFBP2 | 3485 | + |
| IGFBP5 | 3488 | + |

As exemplified in the example section below, a cell population derived from cells cultivated in 3D culture is characterized by differences in expression levels of one or more genes compared to a control population cultured in 2D culture (as indicated in Table 11).

In some embodiments, the cell population of the instant invention is characterized by at least 2 folds change in expression levels of one or more genes listed in table 11. In some embodiments, the cell population of the instant invention is characterized by at least 3 folds change in expression levels of one or more genes listed in table 11. In some embodiments, the cell population of the instant invention is characterized by at least 3 folds decrease in expression levels of one or more genes listed in table 11, comprising: CLDN1, SFRP1, BCYRN, CDCA7, FLJ21986, ODC1, OSR1, LOC100130516, and ROR1. In some embodiments, the cell population of the instant invention is characterized by at least 3 folds decrease in expression levels of one or more genes selected from the group consisting of: CLDN1, SFRP1, BCYRN, CDCA7, FLJ21986, ODC1, OSR1, LOC100130516, and ROR1.

In some embodiments, the cell population of the instant invention is characterized by at least 3 folds increase in expression levels of one or more genes listed in table 11, comprising: ALOX15B, HEPH, FNDC1, C14ORF132, PFKFB4, GABARAPL1, CRISPLD2, C13ORF15, SLC6A10P, JAM2, NBL1, OGN, ASS1, SSPN, ALOX15B, TMEM90B, FLJ35258, TMEM16A, CRLF1, CD24, CMTM8, ARHGEF19, OMD, BTBD11CYGB, C1QTNF5, MARCKSL1, INSC, ATPIB1, CPE, NBL1, ENC1, APCDD1L, SEZ6L2, SLC7A8, ISLR, ATPIB1, TSPAN7, SAMD11, ATPIB1, ALDOC, RGS2, DYNC1I1, RASL11B, EYA2, DIO2, CRYAB, KLK4, MXRA5, CA9, H19, PENK, RARRES2, KANK4, PTGES, and ANKRD38. In some embodiments, the cell population of the instant invention is characterized by at least 3 folds increase in expression levels of one or more genes selected from the group consisting of: ALOX15B, HEPH, FNDC1, C14ORF132, PFKFB4, GABARAPL1, CRISPLD2, C13ORF15, SLC6A10P, JAM2, NBL1, OGN, ASS1, SSPN, ALOX15B, TMEM90B, FLJ35258, TMEM16A, CRLF1, CD24, CMTM8, ARHGEF19, OMD, BTBD11CYGB, C1QTNF5, MARCKSL1, INSC, ATPIB1, CPE, NBL1, ENC1, APCDD1L, SEZ6L2, SLC7A8, ISLR, ATP1B1, TSPAN7, SAMD11, ATP1B1, ALDOC, RGS2, DYNC1I1, RASL11B, EYA2, DIO2, CRYAB, KLK4, MXRA5, CA9, H19, PENK, RARRES2, KANK4, PTGES, and ANKRD38. In some embodiments, the cell population of the instant invention is characterized by at least 4 folds increase in expression levels of one or more genes selected from the group consisting of: FLJ35258, TMEM16A, CRLF1, CD24, CMTM8, ARHGEF19, OMD, BTBD11CYGB, C1QTNF5, MARCKSL1, INSC, ATPIB1, CPE, NBL1, ENC1, APCDD1L, SEZ6L2, SLC7A8, ISLR, ATPIB1, TSPAN7, SAMD11, ATP1B1, ALDOC, RGS2, DYNC1I1, RASL11B, EYA2, DIO2, CRYAB, KLK4, MXRA5, CA9, H19, PENK, RARRES2, KANK4, PTGES, and ANKRD38. In some embodiments, the cell population of the instant invention is characterized by at least 5 folds increase in expression levels of one or more genes selected from the group consisting of: SLC7A8, ISLR, ATPIB1, TSPAN7, SAMD11, ATP1B1, ALDOC, RGS2, DYNC1I1, RASL11B, EYA2, DIO2, CRYAB, KLK4, MXRA5, CA9, H19, PENK, RARRES2, KANK4, PTGES, and ANKRD38.

In some embodiments, the cell population of the instant invention is characterized by at least 6 folds increase in expression levels of one or more genes selected from the group consisting of: ATP1B1, TSPAN7, SAMD11, ATP1B1, ALDOC, RGS2, DYNC1I1, RASL11B, EYA2, DIO2, CRYAB, KLK4, MXRA5, CA9, H19, PENK, RARRES2, KANK4, PTGES, and ANKRD38. In some embodiments, the cell population of the instant invention is characterized by at least 7 folds increase in expression levels of one or more genes selected from the group consisting of: DIO2, CRYAB, KLK4, MXRA5, CA9, H19, PENK, RARRES2, KANK4, PTGES, and ANKRD38. In some embodiments, the cell population of the instant invention is characterized by at least 8 folds increase in expression levels of one or more genes selected from the group consisting of: MXRA5, CA9, H19, PENK, RARRES2, KANK4, PTGES, and ANKRD38. In some embodiments, the cell population of the instant invention is characterized by at least 10 folds increase in expression levels of one or more genes selected from the group consisting of: PENK, RARRES2, KANK4, PTGES, and ANKRD38.

TABLE 11
Differentially expressed gene having significant modulation of expression

| | | Expression | | Entrez | Expression |
|---|---|---|---|---|---|
| | Entrez | relative to | | Gene | relative to |
| Gene Name | Gene ID | control | Gene Name | ID* | control |
| CLDN11 | 5010 | − | C1QTNF5 | 114902 | + |
| SFRP1 | 6422 | − | MARCKSL1 | 65108 | + |
| BCYRN1 | 618 | − | INSC | 387755 | + |
| CDCA7 | 83879 | − | ATP1B1 | 481 | + |
| ODC1 | 4953 | − | CPE | 1363 | + |
| OSR1 | 13097 | − | NBL1 | 4681 | + |
| ROR1 | 4919 | − | ENC1 | 8507 | + |
| ALOX15B | 247 | + | APCDD1L | 164284 | + |
| HEPH | 9843 | + | SEZ6L2 | 26470 | + |
| FNDC1 | 84624 | + | SLC7A8 | 23428 | + |
| C14ORF132 | 56967 | + | ISLR | 3671 | + |
| CYGB | 114757 | + | ATP1B1 | 481 | + |
| PFKFB4 | 5210 | + | TSPAN7 | 7102 | + |
| GABARAPL1 | 23710 | + | SAMD11 | 148398 | + |
| CRISPLD2 | 83716 | + | ATP1B1 | 481 | + |
| RGCC | 28984 | + | ALDOC | 230 | + |
| (C13ORF15) | | | | | |
| SLC6A10P | 386757 | + | RGS2 | 5997 | + |
| JAM2 | 58494 | + | DYNC1I1 | 1780 | + |
| NBL1 | 4681 | + | RASL11B | 65997 | + |
| OGN | 4969 | + | EYA2 | 2139 | + |
| ASS1 | 445 | + | DIO2 | 1734 | + |
| SSPN | 8082 | + | CRYAB | 1410 | + |
| ALOX15B | 247 | + | KLK4 | 9622 | + |
| SYNDIG1 | 79953 | + | MXRA5 | 25878 | + |
| (TMEM90B) | | | | | |
| CRLF1 | 9244 | + | CA9 | 768 | + |
| CD24 | 100133941 | + | H19 | 283120 | + |
| CMTM8 | 152189 | + | PENK | 5179 | + |
| ARHGEF19 | 128272 | + | RARRES2 | 5919 | + |
| OMD | 4958 | + | KANK4 | 163782 | + |
| BTBD11 | 121551 | + | PTGES | 9536 | + |

### Modulation of Expression Following Osteogenic Induction

As exemplified in the examples section below (Table 12), HATDCs subjected to osteogenic induction were found to exhibit a modulation in expression levels of the genes ATOH8, CGB1, CMTM4, FOXO, ID1, ID2, ID3, NEBL, OSR1, PRRX2, SAMD11, SLC16A3, and SMAD9.

In some embodiments, a cell population derived from cells subjected to osteogenic induction is characterized by differences in expression levels of one or more genes selected from the group consisting of: ATOH8, CGB1, CMTM4, FOXO, ID1, ID2, ID3, NEBL, OSR1, PRRX2, SAMD11, SLC16A3, and SMAD9. In some embodiments, a cell population derived from cells subjected to osteogenic induction is characterized by an induction of one or more genes selected from the group consisting of: ATOH8, CGB1, CMTM4, FOXO, ID1, ID2, ID3, NEBL, PRRX2, SAMD11, SLC16A3, and SMAD9. In some embodiments, a heterogeneous cell population derived from cells subjected to osteogenic induction is characterized by reduction of an expression level of the OSR1 gene.

### Method for Identifying Compositions Suitable for Transplantation

According to one aspect, there is provided a method for determining suitability of a cell composition for transplantation, the method comprises determining the expression levels of a plurality of genes or products thereof, of said composition, wherein a significant difference of the expression levels of a plurality of genes compared to a control is an indication of a composition suitable for transplantation.

In some embodiments, the plurality of genes are selected from the genes listed in Tables 1-11. In some embodiments, the plurality is selected from one or more genes of each one of tables 1-11. In some embodiments, the plurality is selected from the genes listed in a Table selected from table 1-11. In some embodiments, the plurality of genes are selected from the genes listed in any one of Table 1-11.

In some embodiment the control population is a population derived from cells cultivated in a 2 dimensional (2D) culture. In some embodiments, the control population is derived from cells cultivated in 2D culture and subjected to an osteogenic induction.

The terms “determining,” “measuring,” “assessing,” and “assaying” are used interchangeably and include both quantitative and qualitative determinations. These terms refer to any form of measurement, and include determining if a characteristic, trait, or feature is present or not. Assessing may be relative or absolute.

According to some embodiments, the plurality of genes comprises at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 55, at least 60, at least 65, at least 70, at least 75, at least 80, at least 85, at least 90, at least 95 different genes listed in Tables 1-10. Each possibility represents a separate embodiment of the instant invention. According to some embodiments, the plurality of genes comprises at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most t 20, at most 21, at most 22, at most 23, at most 24, at most 25, at most 26, at most 27, at most 28, at most 29, at most 30, at most 31, at most 32, at most 33, at most 34, at most 35, at most 36, at most 37, at most 38, at most 39, at most 40, at most 41, at most 42, at most 43, at most 44, at most 45, at most 46, at most 47, at most 48, at most 49, at most 50, at most 55, at most 60, at most 65, at most 70, at most 75, at most 80, at most 85, at most 90, at most 95 different genes listed in Tables 1-11. Each possibility represents a separate embodiment of the instant invention.

The plurality of genes described herein, optionally includes any sub-combination and/or a combination featuring at least one other marker, for example other known genes.

### Determination of Gene Expression

Gene expression is the transcription of DNA into messenger RNA by RNA polymerase. The term “expression” as used herein refers to the biosynthesis of a gene product, including the transcription and/or translation of said gene product. Thus, expression of a nucleic acid molecule may refer to transcription of the nucleic acid fragment (e.g., transcription resulting in mRNA or other functional RNA) and/or translation of RNA into a precursor or mature protein (polypeptide).

Up-regulation describes a gene which has been observed to have higher expression (e.g., higher mRNA levels) in one sample (e.g., a sample suitable for transplantation) compared to another (e.g., a control sample). Down-regulation describes a gene which has been observed to have lower expression (e.g., lower mRNA levels) in one sample (e.g., a sample suitable for transplantation) compared to another (e.g., a control sample).

In an embodiment, the gene expression is measured at the protein levels. Examples of methods to measure the amount/level of a protein in a sample include, but are not limited to: Western blot, immunoblot, enzyme-linked immunosorbent assay (ELISA), “sandwich” immunoassays, radioimmunoassay (RIA), immunoprecipitation, surface plasmon resonance (SPR), chemiluminescence, fluorescent polarization, phosphorescence, immunohistochemical (IHC) analysis, matrix-assisted laser desorption/ionization time-of-flight (MALDI-TOF) mass spectrometry, microcytometry, microarray, antibody array, microscopy (e.g., electron microscopy), flow cytometry, and proteomic-based assays.

In another embodiment, the gene expression is measured at the nucleic acid (mRNA, cDNA) level.

The term “nucleic acid” is well known in the art. A “nucleic acid” as used herein will generally refer to a molecule (i.e., a strand) of DNA, RNA or a derivative or analog thereof, comprising a nucleobase. A nucleobase includes, for example, a naturally occurring purine or pyrimidine base found in DNA (e.g., an adenine “A,” a guanine “G,” a thymine “T” or a cytosine “C”) or RNA (e.g., an A, a G, an uracil “U” or a C). The terms “polynucleotide,” “polynucleotide sequence,” “nucleic acid sequence,” and “nucleic acid molecule” are used interchangeably herein.

Numerous detection and quantification technologies may be used to determine the expression level of the plurality of nucleic acids, including but not limited to: PCR, RT-PCR; RT-qPCR; NASBA; Northern blot technology; a hybridization array; branched nucleic acid amplification/technology; TMA; LCR; High-throughput sequencing or next generation sequencing (NGS) methods such as RNA-seq, in situ hybridization technology; and amplification process followed by HPLC detection or MALDI-TOF mass spectrometry.

In embodiments of the invention, all or part of a nucleic acid may be amplified and detected by methods such as the polymerase chain reaction (PCR) and variations thereof, such as, but not limited to, quantitative PCR (Q-PCR), reverse transcription PCR, and real-time PCR (including as a means of measuring the initial amounts of mRNA copies for each sequence in a sample). Such methods would utilize one or two primers that arc complementary to portions of a nucleic acid, where the primers are used to prime nucleic acid synthesis. The newly synthesized nucleic acids are optionally labeled and may be detected directly or by hybridization to a polynucleotide of the invention. The newly synthesized nucleic acids may be contacted with polynucleotides (containing sequences) under conditions which allow for their hybridization. Additional methods to detect the expression of expressed nucleic acids include RNAse protection assays, including liquid phase hybridizations, and in situ hybridization of cells.

As would be understood by the skilled person, detection of expression of nucleic acids may be performed by the detection of expression of any appropriate portion or fragment of these nucleic acids, or the entire nucleic acids. Preferably, the portions are sufficiently large to contain unique sequences relative to other sequences expressed in a sample. Moreover, the skilled person would recognize that either strand of a nucleic acid may be detected as an indicator of expression of the nucleic acid. This follows because the nucleic acids are expressed as RNA molecules in cells, which may be converted to cDNA molecules for case of manipulation and detection. The resultant cDNA molecules may have the sequences of the expressed RNA as well as those of the complementary strand thereto. Thus either the RNA sequence strand or the complementary strand may be detected. Of course is it also possible to detect the expressed RNA without conversion to cDNA.

In an embodiment, the method comprises performing a reverse transcription of mRNA molecules present in a sample; and amplifying the target cDNA and the one or more control cDNAs using primers hybridizing to the cDNAs.

A common technology used for measuring RNA abundance is RT-qPCR where reverse transcription (RT) is followed by real-time quantitative PCR (qPCR). Commercially available systems for quantitative PCR may be used, for example, “Real Time PCR System” of Applied Biosystems®, LightCycler® from Roche, iCycler® from BioRad®, and others. Reverse transcription first generates a DNA template from the RNA. This single-stranded template is called cDNA. The cDNA template is then amplified in the quantitative step, during which the fluorescence emitted by labeled hybridization probes or intercalating dyes changes as the DNA amplification process progresses. Quantitative PCR produces a measurement of an increase or decrease in copies of the original RNA and has been used to attempt to define changes of gene expression in cancer tissue as compared to comparable healthy tissues (Nolan T, et al. Nat Protoc 1:1559-1582, 2006; Paik S. The Oncologist 12:631-635, 2007; Costa C, et al. Transl Lung Cancer Research 2:87-91, 2013).

Massive parallel sequencing made possible by next generation sequencing (NGS) technologies is another way to approach the enumeration of RNA transcripts in a tissue sample and RNA-seq is a method that utilizes this. It is currently the most powerful analytical tool used for transcriptome analyses, including gene expression level difference between different physiological conditions, or changes that occur during development or over the course of disease progression. Specifically, RNA-seq can be used to study phenomena such as gene expression changes, alternative splicing events, allele-specific gene expression, and chimeric transcripts, including gene fusion events, novel transcripts and RNA editing.

As used herein, the terms “amplification” or “amplify” mean one or more methods known in the art for copying a target nucleic acid, e.g., the genes listed in Tables 1-11, thereby increasing the number of copies of a selected nucleic acid sequence. Amplification may be exponential or linear. In a particular embodiment, the target nucleic acid is RNA.

As used herein, “nucleic acid” refers broadly to segments of a chromosome, segments or portions of DNA, cDNA, and/or RNA. Nucleic acid may be derived or obtained from an originally isolated nucleic acid sample from any source (e.g., isolated from, purified from, amplified from, cloned from, or reverse transcribed from sample DNA or RNA).

As used herein, the term “oligonucleotide” refers to a short polymer composed of deoxyribonucleotides, ribonucleotides or any combination thereof. Oligonucleotides are generally between about 10 and about 100 nucleotides in length. Oligonucleotides are typically 15 to 70 nucleotides long, with 20 to 26 nucleotides being the most common. An oligonucleotide may be used as a primer or as a probe. An oligonucleotide is “specific” for a nucleic acid if the oligonucleotide has at least 50% sequence identity with a portion of the nucleic acid when the oligonucleotide and the nucleic acid are aligned. An oligonucleotide that is specific for a nucleic acid is one that, under the appropriate hybridization or washing conditions, is capable of hybridizing to the target of interest and not substantially hybridizing to nucleic acids which are not of interest. Higher levels of sequence identity are preferred and include at least 75%, at least 80%, at least 85%, at least 90%, or at least 95% sequence identity.

As used herein, a “fragment” in the context of a nucleic acid refers to a sequence of nucleotide residues which hare at least about 5 nucleotides, at least about 7 nucleotides, at least about 9 nucleotides, at least about 11, nucleotides, or at least about 17, nucleotides. A fragment is typically less than about 300 nucleotides, less than about 100 nucleotides, less than about 75 nucleotides less than about 50 nucleotides, or less than about 30 nucleotides. In certain embodiments, the fragments can be used in polymerase chain reaction (PCR), or various hybridization procedures to identify or amplify identical or related DNA molecules.

As used herein, a “primer” for amplification is an oligonucleotide that specifically anneals to a target or marker nucleotide sequence. The 3′ nucleotide of the primer should be identical to the target or marker sequence at a corresponding nucleotide position for optimal primer extension by a polymerase. As used herein, a “forward primer” is a primer that anneals to the anti-sense strand of double stranded DNA (dsDNA). A “reverse primer” anneals to the sense-strand of dsDNA.

As used herein, “target nucleic acid” refers to segments of a chromosome, a complete gene with or without intergenic sequence, segments or portions a gene with or without intergenic sequence, or sequence of nucleic acids to which probes or primers are designed. Target nucleic acids may be derived from genomic DNA, cDNA, or RNA. As used herein, target nucleic acid may be native DNA or a PCR-amplified product.

The detection methods described above are meant to exemplify how the present invention may be practiced and are not meant to limit the scope of invention. It is contemplated that other sequence-based methodologies for detecting the presence of a nucleic acid in a subject sample may be employed according to the invention.

### A Kit for Determining Gene Expression

According to some aspects, the kit, panel or microarray is for determining whether a composition comprising a cell population is suitable for transplantation into a subject in need thereof. In some embodiments, there is provided a kit, panel or microarray comprising multiple ligands, each ligand capable of specifically complexing with, binding to, hybridizing to, or quantitatively detecting or identifying a single gene selected from the genes listed in Tables 1-11. In some embodiments, the multiple ligands are, independently, capable of detecting or identifying a plurality of genes selected from the genes listed in Tables 1-11. In some embodiments, the multiple ligands are, independently, capable of detecting or identifying a plurality of genes selected from the genes listed in a table selected from tables 1-11. The plurality of genes described herein, optionally includes any sub-combination and/or a combination featuring at least one other marker, for example other known genes. In some embodiments, the plurality of genes are selected from: one or more genes selected from table 1, one or more genes selected from table 2, one or more genes selected from table 3, one or more genes selected from table 4, one or more genes selected from table 5, one or more genes selected from table 6, one or more genes selected from table 7, one or more genes selected from table 8, one or more genes selected from table 9, one or more genes selected from table 10, and/or one or more genes selected from table 11, or a combination thereof.

In some embodiments, the kit, panel or microarray comprises at least 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 50, 660, 670, 680, 690, 700, 710, 720, 730, 740, 750, 760, 770, 780, 790, 800, 810, 820, 830, 840, 850, 860, 870, 880, 900, 910, 920, 930, 940, 950, 960, 970, 980, 990, or 1000 different ligands. Each possibility represents a separate embodiment of the instant invention. In some embodiments, the kit, panel or microarray comprises at most 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 50, 660, 670, 680, 690, 700, 710, 720, 730, 740, 750, 760, 770, 780, 790, 800, 810, 820, 830, 840, 850, 860, 870, 880, 900, 910, 920, 930, 940, 950, 960, 970, 980, 990, or 1000 different ligands. Each possibility represents a separate embodiment of the instant invention.

The term “microarray” refers to an ordered arrangement of hybridizable array elements, preferably polynucleotide probes, on a substrate.

In certain embodiments, one or more algorithms or computer programs may be used for comparing the quantified expression levels of each gene in the test sample against a predetermined cutoff (or against a number of predetermined cutoffs). Alternatively, one or more instructions for manually performing the necessary steps by a human can be provided. Algorithms for determining and comparing pattern analysis include, but are not limited to, principal component analysis, Fischer linear analysis, neural network algorithms, genetic algorithms, fuzzy logic pattern recognition, and the like. After analysis is completed, the resulting information can, for example, be displayed on display, transmitted to a host computer, or stored on a storage device for subsequent retrieval.

### Heterogeneous Cell Populations

According to some embodiments, the cell population of the instant invention is a heterogeneous cell population.

As used herein, the term “cell population” refers to a group of at least two cells expressing similar or different phenotypes. In non-limiting examples, a cell population can include at least about 10, at least about 100, at least about 200, at least about 300, at least about 400, at least about 500, at least about 600, at least about 700, at least about 800, at least about 900, at least about 1000 cells expressing similar or different phenotypes. As used herein, the term “heterogeneous cell population” refers to a group of at least two cells wherein at least part of the cells express different phenotypes.

As used herein, the term “mesenchymal stem cell” or “MSC” refers to a cell capable of giving rise to differentiated cells in multiple mesenchymal lineages, specifically to osteoblasts, adipocytes, myoblasts and chondroblasts. Generally, mesenchymal stem cells also have one or more of the following properties: an ability to undergo asynchronous, or symmetric replication that is where the two daughter cells after division can have different phenotypes; extensive self-renewal capacity; and clonal regeneration of the tissue in which they exist, for example, the non-hematopoietic cells of bone marrow. “Progenitor cells” differ from stem cells in that they typically do not have the extensive self-renewal capacity.

In some embodiments, the cell population is a heterogeneous cell population. In some embodiments, the heterogeneous cell population comprises at least 10% cells, at least 20% cells, at least 30% cells, at least 50% cells, at least 50% cells, at least 60% cells, at least 70% cells, at least 80% cells or at least 90% cells having said expression profile described herein.

In some embodiments, the heterogeneous cell population comprises two or more cell types selected from the group consisting of: mesenchymal stem cells, osteoprogenitor cells and osteogenic cells. In some embodiments, 30-70% of cells of the heterogeneous cell population are osteoprogenitor cells. In some embodiments, 40-60% of cells of the heterogeneous cell population are osteoprogenitor cells. In some embodiments, 50-60% of cells of the heterogeneous cell population are osteoprogenitor cells.

In some embodiments, the heterogeneous cell population is derived from cells subjected to osteogenic induction.

The term “osteogenic” or “osteogenesis” refers to proliferation of bone cells and growth of bone tissue (i.e., synthesis and deposit of new bone matrix) from undifferentiated mesenchymal stem cells and cells of osteoblast lineage. Osteogenesis also refers to differentiation or trans-differentiation of progenitor or precursor cells into bone cells (i.e., osteoblasts). Progenitor or precursor cells can be pluripotent stem cells including, e.g., mesenchymal stem cells. Progenitor or precursor cells can be cells pre-committed to an osteoblast lineage (e.g., pre-osteoblast cells) or cells that are not pre-committed to an osteoblast lineage (e.g., pre-adipocytes or myoblasts).

The term “differentiation” as used herein refers to the cellular development of a cell from a primitive stage to a mature formation that is associated with the expression of characteristic set of cell surface antigenic markers. Differentiation is a developmental process whereby cells assume a specialized phenotype, e.g., acquire one or more characteristics or functions distinct from other cell types. In some cases, the differentiated phenotype refers to a cell phenotype that is at the mature endpoint in some developmental pathway (“terminally differentiated cell”).

The term “osteogenic induction” refers to the up-regulation, or stimulation of osteogenic differentiation.

In one embodiment, the heterogeneous cell population is derived from cells that underwent an osteogenic priming period of at least 24 hours. In another embodiment, the heterogeneous cell population is derived from cells that underwent an osteogenic priming period of at least 48 hours. In another embodiment, the heterogeneous cell population is derived from cells that underwent an osteogenic priming period of at least 72 hours. In another embodiment, the heterogeneous cell population is derived from cells that underwent an osteogenic priming period of at least 96 hours.

In some embodiments, induction of osteogenic differentiation of cells is achieved by one or more osteogenic inducers selected from the group consisting of: BMP-2, BMP-3, BMP-4, BMP-5, BMP-6 and BMP-7.

In some embodiments, the cells are treated with at least 25 nano-grams/milliliter of one or more osteogenic inducers to obtain the heterogeneous cell population. In another embodiment, the cells are treated with at least 50 nano-grams/milliliter of one or more osteogenic inducers to obtain the heterogeneous cell population. In another embodiment, the cells are treated with at least 75 nano-grams/milliliter of one or more osteogenic inducers to obtain the heterogeneous cell population. In another embodiment, the cells are treated with at least 100 nano-grams/milliliter of one or more osteogenic inducers to obtain the heterogeneous cell population. In another embodiment, the cells are treated with at least 150 nano-grams/milliliter of one or more osteogenic inducers to obtain the heterogeneous cell population.

For a non-limiting example, the heterogeneous cell population is derived from subjecting cells to osteogenic culture differentiation conditions comprising: osteogenic culture differentiation medium composed of one or more of the following molecules in preferred concentration: dexamethasone (10-200 nM), sodium beta.-glycerophosphate (5-25 mM), 1.25 dihydroxycholecalciferol (calcitriol: 1-50 nM), L-ascorbic acid-2-phosphate (0.05-500 mM) and an osteogenic inducer (10 ng/ml-10 ug/ml).

In another embodiment, the cells are treated with 150 nano-grams/milliliter of one or more osteogenic inducers for 48 hours to obtain the heterogeneous cell population.

In some embodiments, the cells are derived from stem cells. In some embodiments, the stem cells are mesenchymal stem cells (MSCs). In some embodiments, the MSCs are autologous MSCs. In some embodiments, the MSCs are allogenic MSCs. In some embodiments, the autologous MSCs are derived from autologous human adipose tissue, and are referred to as human adipose tissue derived cells (HATDCs). In another embodiment, human adipose tissue derived cells (HATDCs) are cells obtained from adipose tissue by liposuction procedures.

As used herein the term “human adipose tissue derived cells (HATDCs)” refers to a heterogeneous population of cells originated from the vascular stromal compartment of fat tissues which can be used as an alternative cell source for many different cell therapies. As used herein, HATDCs comprise heterogeneous population of cells comprising a plurality of: adipose-derived stem cells (ASC) (CD34− CD45− CD11b−, CD19, HLA−DR−, CD105+, CD73+, CD90+), mesenchymal cells, mesenchymal stem cells, vascular smooth muscle cells (Smooth muscle alpha-actin positive, Desmin positive, h-caldesmon positive, Smooth muscle myosin heavy chain positive), adipogenic, chondrogenic and osteogenic cells in any combination of osteoprogenitors, osteoblasts, osteocytes, chondroblasts, chondrocytes and osteoclasts, as well as endothelial progenitor cells (EPCs) (CD31+ CD34+ CD45− CD144+ CD146+ CD102), hematopoietic progenitor cells (HPCs − CD34+) and mature ECs (CD31+ CD34+ CD45− CD90− CD144+ CD146+ CD105+).

In some embodiments, the cells are cultivated in a 3 dimensional (3D) culture prior to osteogenic induction. In some embodiments, the cells are cultivated in 3D culture on a mineral scaffold. In some embodiments, the cells are cultivated in a 3D culture in a bioreactor or a dynamic growth system. In another embodiment, cells of the invention are maintained and grown at 37ºC in a tissue culture incubator under humidified condition with 5% CO₂.

In some embodiments, the composition comprising the heterogeneous cell population is transplanted in a patient in need thereof. In some embodiments, cells of the heterogeneous cell population of the transplanted composition that were derived ex-vivo, are exposed to in-vivo osteogenic inducers available at the transplantation site (e.g., bone). In some embodiments, the cells of the heterogeneous cell population are further differentiate into mature osteoblasts in-vivo.

As used herein the term “ex-vivo” refers to a process in which cells are removed from a living organism and are propagated outside the organism. As used herein the term “in-vivo” refers to any process that occurs inside a living organism.

In another embodiment, the invention provides a kit comprising: a mineral particle comprising a 3D cell culture attached thereto and instructions for generating the heterogeneous cell population of the invention from the 3D cell culture provided. In another embodiment, the 3D cell culture comprises HATDCs. In another embodiment, the instructions include recommended conditions for osteogenic induction of HATDCs cultivated in 3D culture on a mineral scaffold in order to obtain the composition of the invention. In another embodiment, the kit further provides at least one osteogenic inducer and/or osteogenic culture differentiation medium.

### Multi-Layer Cell Culture

In some embodiments, a multi-layered cell culture is a heterogeneous cell culture composed of at least two cell types. In another embodiment, a multi-layered cell culture is a heterogeneous cell culture composed of at least three cell types. In another embodiment, a multi-layered cell culture is a heterogeneous cell culture composed of at least four cell types. In another embodiment, a multi-layered cell culture comprises human adipose tissue derived cells (HATDCs) 48 hours subsequent to osteogenic priming period.

In another embodiment, a multi-layered cell culture comprises a bottom layer of cells and a top layer of cells. In another embodiment, a multi-layered cell culture comprises a bottom layer of cells, a middle layer of cells and a top layer of cells. In another embodiment, a multi-layered cell culture is a 3D (three dimensional) cell culture (as opposed to a single layer of cells that is termed a 2D (two dimensional) cell culture). In another embodiment, a 3D cell culture consists cells and extra cellular matrix. In another embodiment, a 3D cell culture is grown on the surface of a mineral particle as described herein. In another embodiment, a 3D cell culture consists a biotic matter. In another embodiment, a 3D cell culture of 2 or more cell layers is attached to the mineral particle. In another embodiment, a 3D cell culture of 2 or more cell layers is operably attached to the mineral particle.

In some embodiments, a multi-layered cell culture or a 3D cell culture includes at least 2 layers of cells, wherein at least 10% of the cells in one layer are in contact with at least 10% of the cells in another layer. In some embodiments, a multi-layered cell culture or a 3D cell culture includes at least 3 layers of cells.

In some embodiments, at least 10% of the cells in one layer within a multi-layered cell culture or a 3D cell culture are in contact with at least 10% of the cells in another layer within the same multi-layered cell culture or 3D cell culture. In some embodiments, at least 20% of the cells in one layer within a multi-layered cell culture or a 3D cell culture are in contact with at least 20% of the cells in another layer within the same multi-layered cell culture or 3D cell culture. In some embodiments, at least 30% of the cells in one layer within a multi-layered cell culture or a 3D cell culture are in contact with at least 30% of the cells in another layer within the same multi-layered cell culture or 3D cell culture. In some embodiments, at least 40% of the cells in one layer within a multi-layered cell culture or a 3D cell culture are in contact with at least 40% of the cells in another layer within the same multi-layered cell culture or 3D cell culture. In some embodiments, at least 50% of the cells in one layer within a multi-layered cell culture or a 3D cell culture are in contact with at least 50% of the cells in another layer within the same multi-layered cell culture or 3D cell culture. In some embodiments, at least 60% of the cells in one layer within a multi-layered cell culture or a 3D cell culture are in contact with at least 60% of the cells in another layer within the same multi-layered cell culture or 3D cell culture. In another embodiment, the phrase “in contact” is in physical contact. In another embodiment, the phrase “in contact” is in cell to cell interaction.

In another embodiment, the phrase “3D cell culture” or “3D culture” refers to a culture in which the cells are disposed to conditions which are compatible with cell growth while allowing the cells to grow in more than one layer. In another embodiment, cells within the 3D cell culture are held in a complex network of extra cellular matrix nanoscale fibers that allows the establishment of various local microenvironments. In another embodiment, extra cellular ligands within the ECM mediate not only the attachment to the basal membrane but also access to a variety of vascular and lymphatic vessels. In another embodiment, cells within the 3D cell culture are exposed to oxygen, hormones and nutrients. In another embodiment, a 3D cell culture is characterized by cell-cell and cell-ECM interactions.

### The Scaffold

In another embodiment the composition further comprises osteoconductive particles. As used in here “osteoconductive” refers to the ability of a substance to serve as a suitable template or substance along which bone may grow. For a non-limiting example one or more types of the osteoconductive particles are osteoconductive ceramic particles selected from the group consisting of: calcium carbonate, hydroxyapatite (HA), demineralized bone material, morselized bone graft, cortical cancellous allograft, cortical cancellous autograft, cortical cancellous xenograft, tricalcium phosphate, coralline mineral and calcium sulfate.

In another embodiment, the composition further comprises a mineral particle. In another embodiment, a mineral particle is a scaffold carrying a 3D cell culture. In another embodiment, mineral particle is biocompatible. In another embodiment, cells may attach to the mineral particle. In another embodiment, the mineral particle facilitates expansion of attached cells. In another embodiment, mineral particles are in the form of a pulverized composition. In another embodiment, mineral particles are in the form of a micro-pulverized composition. In another embodiment, mineral particles comprise edges and grooves which provide more cell attachment sites. As used herein, “expansion” or “expanding” refers to a process of cell proliferation substantially devoid of cell differentiation. Cells that undergo expansion hence maintain their cell renewal properties i.e., increase of a cell population (e.g., at least 2 fold) without differentiation accompanying such increase.

In another embodiment, a mineral particle is a bone fiber. In another embodiment, a bone fiber of the invention has enhanced cell-binding surface. In another embodiment, a bone fiber of the invention is derived from a bone tissue. In another embodiment, a bone tissue is cut along its length or along the grain direction of the bone tissue to form a bone fiber.

In another embodiment, a mineral particle is a bone scaffold carrying a 3D cell culture. In another embodiment, a mineral particle is a bone mineral particle. In another embodiment, a mineral particle is a ground mineralized cortical bone. In another embodiment, a mineral particle is a ground mineralized cancellous bone. In another embodiment, a mineral particle is a mineralized cancellous particle. In another embodiment, a mineral particle is a mineralized cortical particle. In another embodiment, a mineral particle is a coral mineral particle. In another embodiment, a mineral particle consists minerals. In another embodiment, a mineral particle comprises calcium phosphate. In another embodiment, a mineral particle comprises a calcium phosphate derivative. In another embodiment, a mineral particle comprises calcium sulfate. In another embodiment, a mineral particle comprises a calcium sulfate derivative. In another embodiment, a mineral particle comprises calcium hydroxyapatite. In another embodiment, a mineral particle comprises a silicate. In another embodiment, a mineral particle comprises a calcium sulfate derivative. In another embodiment, a mineral particle comprises a silicate mineral hydroxyapatite. In another embodiment, a mineral particle comprises beta-3 calcium phosphate. In another embodiment, a mineral particle comprises any combination of minerals known to one of skill in the art.

In some embodiments, the scaffold further comprises extracellular matrix proteins such as fibronectin, laminin, fibrinogen and collagen. In some embodiments, the mineral particle is coated by extracellular matrix proteins.

In another embodiment, a mineral particle has a diameter of at least 50 microns. In another embodiment, a mineral particle has a diameter of at least 100 microns. In another embodiment, a mineral particle has a diameter in the range of 50 microns to 2000 microns. In another embodiment, a mineral particle has a diameter in the range of 100 microns to 1000 microns. In another embodiment, a mineral particle has a diameter in the range of 200 microns to 2000 microns. In some embodiments, the mineral particle has a size of 1 centimeter to 15 centimeters (cm) in length. In some embodiments, the mineral particle has a size of 5 cm to 15 centimeters (cm) in length. In some embodiments, the mineral particle has a size of up to 15 centimeters in length.

In another embodiment, a 3D cell culture attached to mineral particles is grown and/or maintained with cell culture media for a period of 5 days prior to induction of osteogenic differentiation. In another embodiment, a 3D cell culture attached to mineral particles is grown and/or maintained with cell culture media for a period of 4 to 6 days prior to induction of osteogenic differentiation. In another embodiment, a 3D cells culture attached to mineral particles is grown and/or maintained with cell culture media for a period of 2 to 21, or alternatively 4 to 21, or alternatively 2 to 16, or alternatively 3 to 16, or alternatively 4 to 16, or alternatively 1 to 10, or alternatively 2 to 10, or alternatively 3 to 10, or alternatively 4 to 10, or alternatively 1 to 6, or alternatively 2 to 6, or alternatively 3 to 5, or alternatively 3 to 6, or alternatively 4 to 6 days prior to induction of osteogenic differentiation.

### Seeding of Cells

Seeding Cells Migrating from a Tissue

In some embodiment, seeding of cells is carried out by maintaining the adipose tissue in contact with the mineral particles in a specific ratio of tissue to mineral particles for a predefined period of time to allow migration and attachment of cells to the mineral particles. In some embodiments, the adipose tissue is remained intact or alternatively is mechanically dissociated (e.g., minced to small tissue fragments).

In some embodiments, the adipose tissue and the scaffold are maintained in contact for a duration required to facilitate migration of HATDCs from the adipose tissue onto the scaffold and to populate the surface of the scaffold. In some embodiments, at least 3, at least 4, at least 5, at least 6, at least 7, at least 8, at least 9 or at least 10 days incubation in contact are required to facilitate HATDCs to migrate and populate the surface of the mineral scaffold. In some embodiments, the seeding period is at least 3 days. In another embodiment, the seeding period is at least 4 days. In another embodiment, the seeding period is at least 5 days. In another embodiment, the seeding period is at least 6 days. In another embodiment, the seeding period is at least 7 days. In some embodiments, the adipose tissue and the scaffold are maintained in contact for 3-7 days. In some embodiments, the adipose tissue and the scaffold are maintained in contact for 3-10 days. In some embodiments, the adipose tissue and the scaffold are maintained in contact for 5-10 days.

In some embodiments, the adipose tissue and the scaffold have a ratio of 1 microliter tissue per 1 milligram scaffold, herein after referred to as a ratio of 1:1. In some embodiments, the ratio ranges from 10:1-1:10, 9:1-1:9, 8:1-1:8, 7:1-1:7, 6:1-1:6, e5e5, 4:1-1:4, 3:1-1:3, or 2:1-1:2 respectively. In some embodiments, the adipose tissue and the scaffold have a ratio ranging from 3:1-1:2, respectively. In some embodiments, the adipose tissue and the scaffold have a ratio ranging from 2:1-1:4 respectively. In some embodiments, the ratio ranges from 2:1-1:3, 2:1-1:2, 3:1-1:4, 1:1-1:4, 1:1-1:2 or 2:1-1:1 respectively. Each possibility represents a separate embodiment of the present invention. In some embodiments, the ratio between the adipose tissue and the scaffold is 1:1. For a non-limiting example, 1 milliliter adipose tissue is contacted with 1 gram mineral scaffold.

In some embodiments, to achieve contact between the adipose tissue and the scaffold, the adipose tissue and the scaffold are first mixed in the presence of a medium (e.g., xeno free medium). In some embodiments, the adipose tissue and the scaffold are placed in contact while exposed to media and oxygen. In some embodiments, contacting the adipose tissue and the scaffold allows physical contact of at least a portion of the adipose tissue with at least a portion of the scaffold. For a non-limiting example, contacting may be performed in a vessel, a bioreactor, a plate. In some embodiments, the combined thickness of the adipose tissue in contact with the mineral particles is partially covered by the medium. For a non-limiting example, combined thickness of 1-2 millimeters is maintained in a medium level of 1-2 millimeters. In some embodiments, the culture medium is a xeno-free growth medium. As used herein, “xeno-free” means cell culture conditions free of any cell or cell product of species other than that of the cultured cell. In other embodiments, the media is supplemented with serum. Non-limiting examples of serums include: fetal calf serum (FCS), human AB serum, and autologous serum or platelet lysate.

Seeding Cells that were First Obtained from a Tissue

In other embodiments, seeding of cells is carried out by maintaining a specific concentration of cells in the presence of mineral particles in a specific ratio of cells to mineral particles for a predefined period of time to allow attachment of cells to the mineral particles.

In some embodiments, the attachment period is at least 1 hour. In another embodiment, the attachment period is at least 2 hours. In another embodiment, the attachment period is at least 3 hours. In another embodiment, the attachment period is at least 4 hours. In another embodiment, the attachment period is at least 5 hours. In another embodiment, the seeding period is at least 10 hours. In another embodiment, the seeding period is up to 7 days.

In some embodiments, at least 1×10² cells as described herein are seeded per 1 milligram (mg) of mineral particle. In another embodiment, at least 1×10³ cells as described herein are seeded per 1 mg of mineral particle. In another embodiment, at least 1×10² to 1×10⁶ cells as described herein are seeded per 1 mg of mineral particle. In another embodiment, at least 1×10² to 1×10⁴ cells as described herein are seeded per 1 mg of mineral particle. In another embodiment, at least 5×10² to 5×10⁴ cells as described herein are seeded per 1 mg of mineral particle. In another embodiment, at least 3.5×10³ cells as described herein are seeded per 1 mg of mineral particle.

In some embodiments, the cells as described herein are seeded in a concentration of at least 1×10³ cells per 1 milliliter of culture medium. In another embodiment, the cells as described herein are seeded in a concentration of at least 10×10³ cells per 1 milliliter of culture medium. In another embodiment, the cells as described herein are seeded in a concentration of at least 50×10³ cells per 1 milliliter of culture medium. In another embodiment, the cells as described herein are seeded in a concentration of at least 100×10³ cells per 1 milliliter of culture medium. In some embodiments, the culture medium is a xeno-free growth medium. In other embodiments, the media is supplemented with serum such as fetal calf serum (FCS), human AB serum, and autologous serum or platelet lysate.

### Biotic Components

In another embodiment, the invention provides that the composition further comprises albumin. In another embodiment, the invention provides that the composition further comprises an Extra-Cellular Matrix (ECM) protein. In another embodiment, the invention provides that the composition further comprises fibrin. In another embodiment, the invention provides that the composition further comprises fibronectin. In another embodiment, the invention provides that the composition further comprises collagen type I. In another embodiment, the invention provides that the composition further comprises laminin. In another embodiment, the invention provides that the composition further comprises vitronectin.

In another embodiment, the invention provides that the composition further comprises a Bone Morphogenetic Protein (BMP). In another embodiment, the invention provides that the composition further comprises insulin like growth factor. In another embodiment, the invention provides that the composition further comprises interleukin-1, interleukin-6, a Tumor Necrosis Factor (TNF), RANKL, or any combination thereof. In another embodiment, a composition includes an autologous multicellular 3D cell culture suspended in Human Serum Albumin (HSA) containing medium. In another embodiment, a composition as described herein further comprises an anti-inflammatory agent. In another embodiment, a composition as described herein further comprises an antibiotic.

In another embodiment, the invention provides that the composition further comprises a biocompatible binder. In another embodiment, the biocompatible binder is one or more selected from the group consisting of fibrin adhesive, fibrinogen, thrombin, mussel adhesive protein, silk, elastin, collagen, casein, gelatin, albumin, keratin, chitin and chitosan. In another embodiment, the biocompatible binder are one or more selected from the group consisting of starch, polylactic acid, polyglycolic acid, polylactic-co-glycolic acid, polydioxanone, polycaprolactone, polycarbonate, polyoxoester, polyamino acid, poly-anhydride, polyhydroxybutylate, polyhydroxyvalerate, poly(propylene glycol-co-fumaric acid), tyrosine-based-polycarbonate, polyvinylpyrrolidone, cellulose, ethyl cellulose and carboxy methyl cellulose.

In another embodiment, the invention provides that the composition further comprises vitamins. In another embodiment, the invention provides that the composition further comprises a glucosamine. In another embodiment, the invention provides that the composition further comprises a cytokine. In another embodiment, the invention provides that the composition further comprises growth factors.

In another embodiment, the invention provides that the composition further comprises hyaluronic acid. In another embodiment, the term “Hyaluronic Acid (HA)” is synonymous with hyaluronan or sodium hyaluronate. In another embodiment, hyaluronic acid is within a composition comprising a physiological buffer. In another embodiment, hyaluronic acid has a molecular weight of 200,000 to 850,000 Daltons.

In another embodiment, Hyaluronic acid is a composition for suspending the heterogeneous cell population deposited or attached to the mineral particles. In another embodiment, Hyaluronic acid is a composition comprising from 0.5 mg to 50 mg Hyaluronic acid per 1 mL of solution (comprising a buffer). In another embodiment, Hyaluronic acid composition for suspending cells deposited or attached to the mineral particles is a composition comprising from 0.5 mg to 5 mg Hyaluronic acid per 1 mL of solution (comprising a buffer). In another embodiment, Hyaluronic acid composition for suspending cells deposited or attached to the mineral particles is a composition comprising from 5 mg to 20 mg Hyaluronic acid per 1 mL of solution (comprising a buffer). In another embodiment, Hyaluronic acid composition for suspending cells deposited or attached to the mineral particles is a composition comprising from 10 mg to 30 mg Hyaluronic acid per 1 mL of solution (comprising a buffer). In another embodiment, Hyaluronic acid composition for suspending cells deposited or attached to the mineral particles is a composition comprising from 10 mg to 25 mg Hyaluronic acid per 1 mL of solution (comprising a buffer). In another embodiment, Hyaluronic acid composition for suspending cells deposited or attached to the mineral particles is a composition comprising from 0.05% to 5% by weight Hyaluronic acid. In another embodiment, Hyaluronic acid composition for suspending cells deposited or attached to the mineral particles is a composition comprising from 0.1% to 1% by weight Hyaluronic acid. In another embodiment, Hyaluronic acid composition for suspending cells deposited or attached to the mineral particles is a composition comprising from 0.1% to 0.5% by weight Hyaluronic acid.

In another embodiment, Hyaluronic acid composition for suspending cells deposited or attached to the mineral particles is a solution. In another embodiment, Hyaluronic acid composition for suspending cells deposited or attached to the mineral particles is a gel.

### Process of Making the Bone Repair Composition

The composition of the instant invention may be manufactured by several alternative processes. In some embodiments, the process comprises culturing the cells in a 3D culture. In some embodiments, the process comprises culturing the cells in a 2D culture prior to culturing in a 3D culture.

In some embodiments, cells are first isolated from a tissue sample. In some embodiments, isolation includes plasma removal, centrifugation and/or collagenase incubation. In other embodiments, cells migrate directly from the tissue to the scaffold. In some embodiments, the tissue is an adipose tissue. In some embodiments, the cells are stem cells. In some embodiments, the stem cells are human adipose tissue derived cells.

In some embodiment, isolated cells are first cultivated and expanded in a 2D system (e.g., flask). Next, cells grown in a 2D system are cultivated and expanded ex vivo under sterile conditions on the mineral particles, using a media that allows the attachment and growth of adherent cells. In some embodiments, the media is a xeno-free media. In other embodiments, the media is supplemented with a serum. In some embodiments, culture medium that supported the initial growth and expansion phase of these cells may optionally be replaced by another cell culture formula that supports the differentiation of these cells and bone formation.

In some embodiments a tissue and a scaffold are contacted, wherein the contacting facilitates migration of cells from the adipose tissue onto the scaffold and attachment of the cells thereto, thereby providing a scaffold populated with cells. In some embodiments, the method further comprises the step of culturing and expanding the scaffold populated with cells so as to permit expansion of the cells. In some embodiments, the method comprises a preliminary step of separating the adipose tissue from other cells such as erythrocytes. As used herein, the term “preliminary” refers to a step taken prior to the contacting of the tissue and the scaffold. In some embodiments, separation is utilized by subjecting the adipose tissue to washing such as in saline (e.g., normal saline, phosphate buffered saline (PBS) or cell growth media) followed by centrifugation which results in a pellet containing erythrocytes, debris etc. In some embodiments, the method further comprises a step of separating the adipose tissue from the scaffold and HATDCs attached thereto. In some embodiments, the contact between the adipose tissue and the scaffold populated by HATDCs may be detached such as by mixing resulting in a precipitated scaffold populated with HATDCs and a floating adipose tissue. In some embodiments, separation is achieved by removing the adipose tissue. In some embodiments, the floating adipose tissue is removed by washing (e.g. with media). For a non-limiting example, separation may be achieved by aspirating liquids (e.g., by pipetting) and mixing (e.g., by vortexing) the adipose tissue and the scaffold resulting in a precipitated scaffold populated with HATDCs and a floating adipose tissue and, which can be easily removed.

In another embodiment, the 3D heterogeneous cell population attached to the mineral particle is derived from a 3D cell culture attached to the mineral particles subjected to flow-through bioreactor system. In another embodiment, the 3D heterogeneous cell population attached to the mineral particle is derived from further subjecting the 3D cell culture to osteogenic differentiation. In another embodiment, the 3D cell culture are subjected to osteogenic differentiation for 48 hours, or alternatively at least 24 hours, or alternatively at least 48 hours, or alternatively at least 72 hours, or alternatively at least 96 hours.

In another embodiment, the growth medium (cell media) is supplemented with growth factors and cytokines, such as, for example, one or more of: Transforming Growth Factor beta (TGF beta), Insulin-like Growth Factor-1 (IGF-1), Osteogenic protein-1 (OP-1), Fibroblast Growth Factor (FGF) members such as FGF-2, FGF-9 and FGF-10 and members of Bone Morphogenic Proteins (BMP) such as BMP-2, BMP-3, BMP-4, BMP-5, BMP-6 and BMP-7.

In another embodiment, the mineral particle covered by a 3D culture of the heterogeneous cell population is transplanted into a subject in need thereof. In another embodiment, a mineral particle covered by a 3D culture of the heterogeneous cell population is transplanted into a pre-determined site of bone loss or gap.

In another embodiment, the implantable composition of the invention is provided with a syringe. In another embodiment, there is provided herein: a syringe, the 3D heterogeneous cell population of the invention deposited or attached to the mineral particles, and semisolid media (e.g., hyaluronic acid). In another embodiment, provided herein a kit comprising: a syringe, a suspension comprising: the 3D heterogeneous cell population deposited or attached to the mineral particles mineral particles suspended in semisolid media.

In another embodiment, a pharmaceutical composition for filing a gap within a bone is produced by simply mixing semisolid media (e.g., hyaluronic acid) and the 3D heterogeneous cell population attached the mineral particles of the invention. In another embodiment, the pharmaceutical composition for filing a gap within a bone is produced by simply mixing semisolid media and a suspension comprising: the 3D heterogeneous cell population deposited or attached to the mineral particles mineral particles suspended in cell culture media.

In another embodiment, a kit for filing a gap within a bone, comprises a first part that contains an effective amount of semisolid media, and a second part that contains an effective amount of a suspension comprising: the 3D heterogeneous cell population deposited or attached to the mineral particles mineral particles suspended in cell culture media. In another embodiment, the kit is for injection, and the first and second parts can be in solution form and are separately placed in independent packs (such as plastic bottles or glass bottles like ampoules). In another embodiment, each pack can comprise multiple dosages, but preferably a single dosage, of the first or second part. In another embodiment, prior to injection, the two parts are put into the injection syringe according to the information in the instruction (comprising the information such as the operation method of the kit, the mixing ratio of the solutions, etc.) to apply the formulation. In another embodiment, prior to injection, the two parts are put into a mixing means inside or outside the syringe. In another embodiment, prior to injection, the two parts are mixed by a mixing means inside or outside the syringe.

The term semi-solid refers to materials having a gel-like consistency, such as for a non-limiting example, being substantially dimensionally stable at room temperature, but have a certain elasticity and flexibility, typically due to a residual solvent content.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

In the discussion unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the specification and claims is considered to be the inclusive “or” rather than the exclusive or, and indicates at least one of, or any combination of items it conjoins.

It should be understood that the terms “a” and “an” as used above and elsewhere herein refer to “one or more” of the enumerated components. It will be clear to one of ordinary skill in the art that the use of the singular includes the plural unless specifically stated otherwise.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about”. Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

In the description and claims of the present application, each of the verbs, “comprise”, “include” and “have” and conjugates thereof, are used to indicate that the object or objects of the verb are not necessarily a complete listing of components, elements or parts of the subject or subjects of the verb. Other terms as used herein are meant to be defined by their well-known meanings in the art.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Additional objects, advantages, and novel features of the present invention will become apparent to one ordinarily skilled in the art upon examination of the following examples, which are not intended to be limiting. Additionally, each of the various embodiments and aspects of the present invention as delineated herein above and as claimed in the claims section below finds experimental support in the following examples.

### EXAMPLES

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, C T (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, C A (1990); Marshak et al., “Strategies for Protein Purification and Characterization—A Laboratory Course Manual” CSHL Press (1996); all of which are incorporated by reference. Other general references are provided throughout this document.

### Materials and Methods:

### Experimental Design

Cells were cultivated under four different conditions (denoted as treatment groups: BL, A, B, and C), these different conditions are summarized in table 13. Each experiment was repeated three times.

Group BL represents a 2D culture maintained for up to 4 passages, and expression levels of genes in this group were used as base line levels of gene expressions. HATDCs were cultured in 2D system with xeno free medium for 2-4 passages. BMP2 was not supplemented to the medium. As used herein, the term “passage” refers to a cell culture technique in which cells growing in culture that have attained confluence or are close to confluence in a tissue culture vessel are removed from the vessel, diluted with fresh culture media (i.e. diluted 1:5) and placed into a new tissue culture vessel to allow for their continued growth and viability.

Group A, HATDCs were cultured in 2D system with xeno free medium. Following 1-3 passages, cells were reseeded in 2D system and 1-2 days after seeding (day 0) the growth medium was supplemented with one or more osteogenic inducers and cells were cultured for additional two days (Day 2, group A).

Group B, HATDCs were cultured in flasks (2D) with xeno-free medium for 1-2 passages. Next, cells were seeded in 3D system on cortical scaffold using xeno-free medium. Following 4-5 days from seeding in 3D, cells were supplemented with one or more osteogenic inducers to induce osteogenic differentiation and cultured for additional two days (Day 2, System B) until harvesting.

Group C, adipose tissue is placed on mineral scaffold in xeno-free medium, and HATDCs are migrating to the scaffold particles. Following 10-12 days from seeding, cells were supplemented with one or more osteogenic inducers to induce osteogenic differentiation and cultured for additional two days.

HADTCs cultured in 2D were harvested at day 0 (before BMP2 induction) and on day 2 post osteogenic induction (Day 2). HADTCs cultured in 3D (Groups B and C) were harvested 2 days post osteogenic induction (Day 2).

The osteogenic induction is induced by one or more osteogenic inducers such as BMP-2, BMP-3, BMP-4, BMP-5, BMP-6 and BMP-7

TABLE 13
Denotes the four groups cultivated under different conditions

| Treatment group | Conditions |
|---|---|
| BL (Baseline) | 2D system, without osteogenic induction |
| A | 2D system, 2 days osteogenic induction |
| B | 3D system, 2 days osteogenic induction |
| C | 3D system, 2 days osteogenic induction |

### RNA Sample Preparation:

RNA was extracted using the Qiacube robot with RNeasy mini kit (Qiagen). The quality of all total RNA samples was evaluated using TapeStation (Agilent). The RNA value of all samples was >9.5. RNA was amplified into biotinylated cRNA by in vitro transcription using the TargetAmp Nano labeling kit for Illumina BeadChips (Epicentre). Biotinylated cRNAs was hybridized to an Illumina HumanHT-12 v4 Expression BeadChip according to the Direct Hybridization assay (Illumina Inc.). The hybridized chips was stained with streptavidin with streptavidin-Cy3 (GE Healthcare Amersham), scanned with Illumina HiScan and images were imported into GenomeStudio (Illumina) for quality control (QC). The data was then imported to JMP Genomics (SAS) for statistical analysis and to IPA for network enrichment analysis.

### Microarray

For microarray analysis, HumanHT-12 v4 Expression BeadChip Kit (illumina), which targets more than 47,000 probes, was used. Raw data obtained contained more than 47,000 probes. Following log 2 transformation, filtration for low expression and filtration for low variability between samples, about 9,000 probes were retained for statistical analysis.

### Statistical Analysis:

The raw gene expression data was exported from GenomeStudio and imported into JMP Genomics v7 software (SAS Institute Inc, Cary, NC). Quality control and analysis in JMP Genomics was done on log 2 transformed data, after filtering for non-expressed genes (detection p-value<0.01), and for low variance transcripts across samples (variance <5%). Data Distribution showed similar expression, therefore that data was not normalized. The data was analyzed using one-way ANOVA. Differently expressed genes (DEGs) were defined as transcripts that were statistically significant at corrected p-value≤0.05 using the False Discovery Rate (FDR) with at least two-fold change differences. Data analysis was done using the following software: (1) GeneAnalytics, LifeMap sciences, (2) Ingenuity Pathway analysis (IPA 8.0), Ingenuity, Qiagen.

### Example 1

Expression of Endogenous BMP-2, SP7, ALP as Analyzed by qRT-PCR

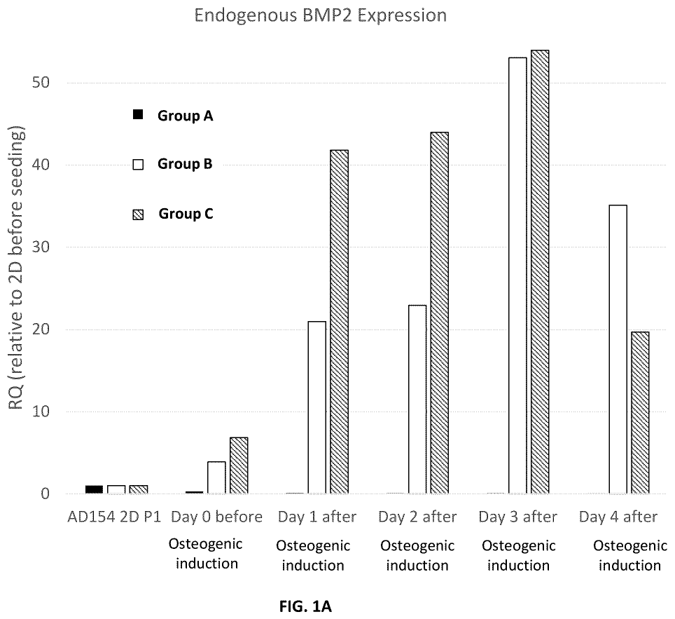

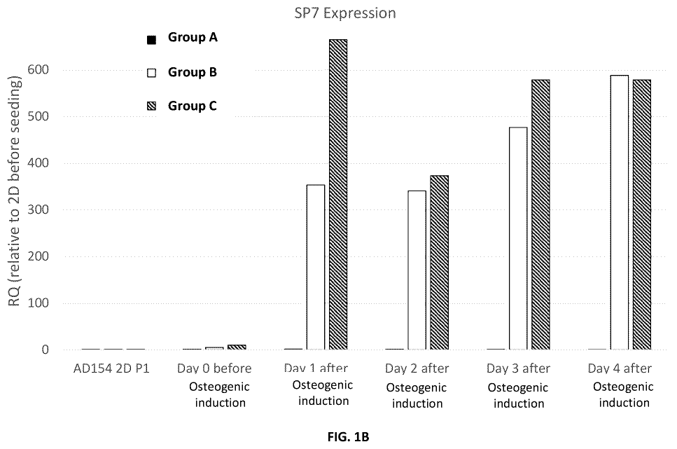

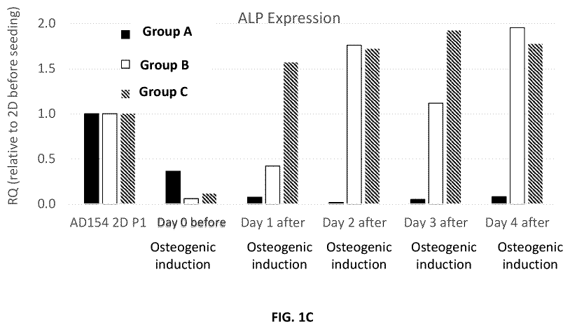

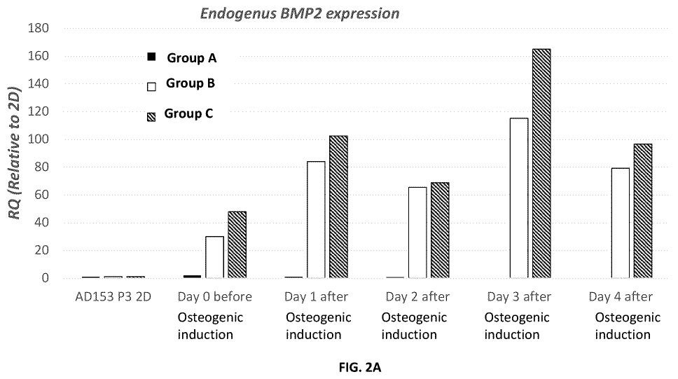

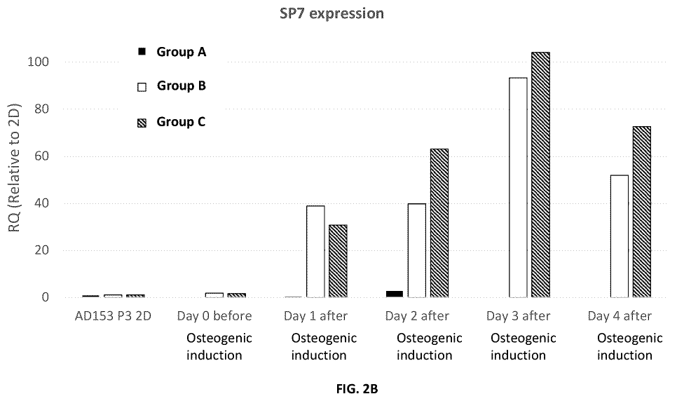

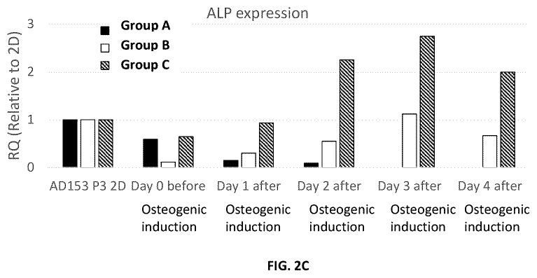

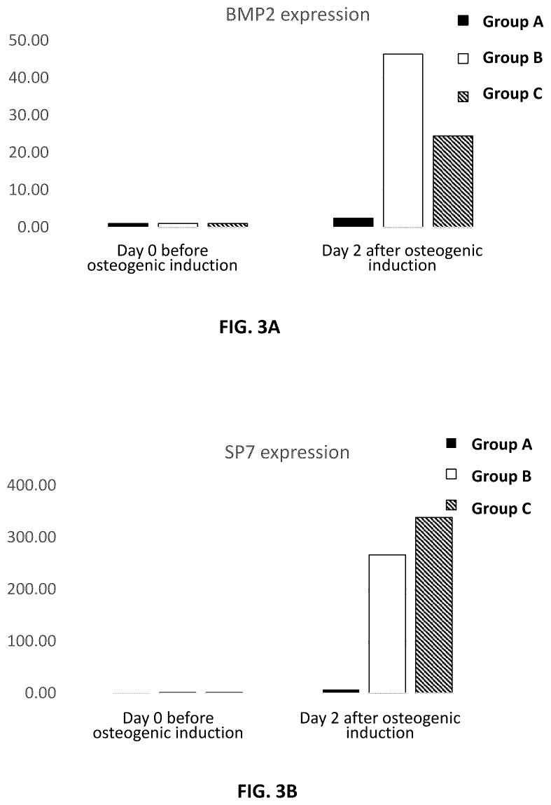

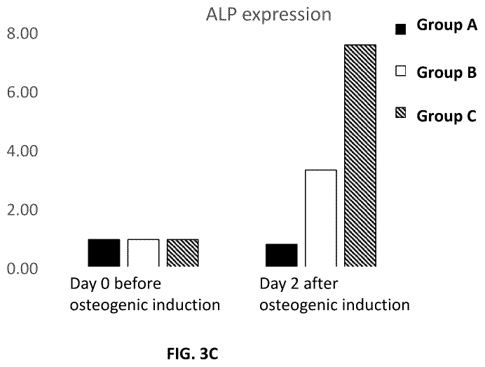

In order to evaluate the osteogenic potentials of the 3D systems (groups C and D) and the 2D system (group A), expression levels of the early osteogenic markers (endogenous bone morphogenetic protein (BMP2), Ostrix (SP7), and Alkaline-Phosphatase (ALP)), were examined. First, osteogenic differentiation of groups A, B, and C, was induced by 150 ng/ml BMP2 supplemented to xeno-free medium, for two days. Next, RNA samples, obtained from systems A, B, and C, were analyzed by qRT-PCR. Expression levels were analyzed relative to that of the control group (group BL). The experiment was conducted using three replicates from different biological sources (denoted AD153, AD154, and AD160), results are represented in FIGS. 1, 2, and 3, respectively.

The expression levels of early osteogenic markers: BMP-2 (FIGS. 1A, 2A and 3A), SP7 2 (FIGS. 1B, 2B and 3B) and ALP 2 (FIGS. 1C, 2C and 3C) were elevated following osteogenic induction.

Previous studies have demonstrated that endogenous expression of BMP2 plays a critical role and is essential for osteogenic differentiation, in addition to the exogenous BMP2. Results demonstrate the elevation of endogenous BMP2 in the 3D-HADTCs, induced by exogenous BMP2 (FIGS. 1C, 2C and 3C). The endogenous BMP2 was increased by an average (for the three batches that were used) of 3.02±0.76 folds at day 3 of differentiation.

Notably, the expression of all tested genes; BMP2, SP7, ALP, was higher in 3D post BMP2 treatment compared to 2D with or without BMP2 (FIGS. 1A-C, 2A-C, and 3A-C). These results indicate for the increased potency of HADTCs, grown in 3D systems, to differentiate into osteoblasts, relative to HADTCs grown in 2D system.

### Example 2

### Microarray Analysis

First the variance component was established by analyzing the difference between different treatments groups (systems) and between different biological replicates (AD153, AD154, and AD160).

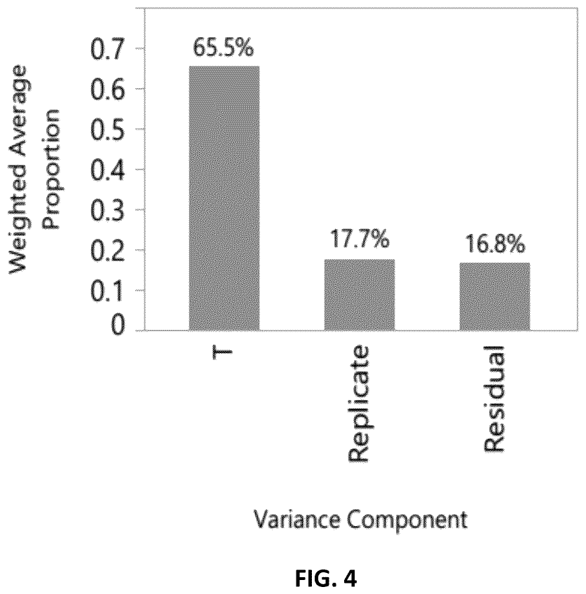

Results demonstrate that about 66% of the total variance is due to difference between treatment groups, and an additional ˜18% of the variance is due to difference between biological replicates (FIG. 4).

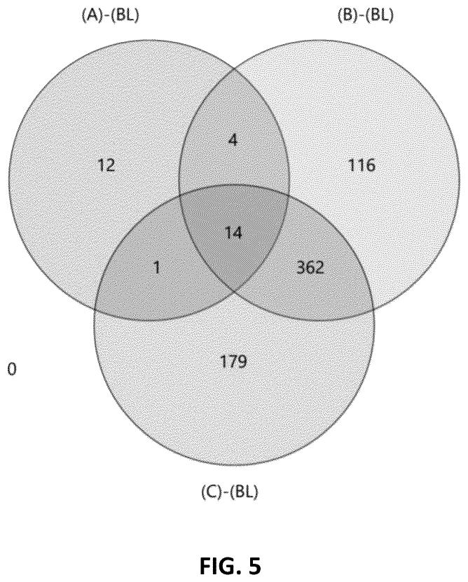

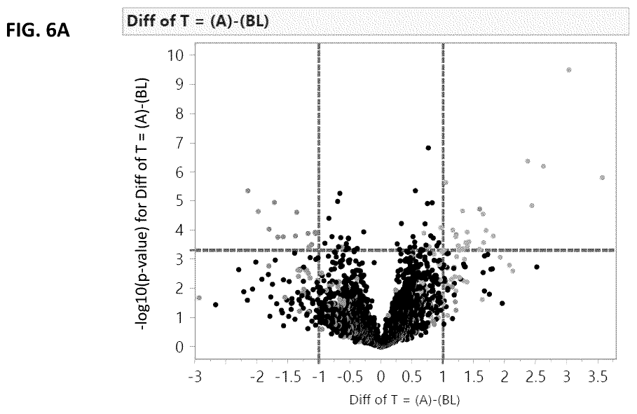

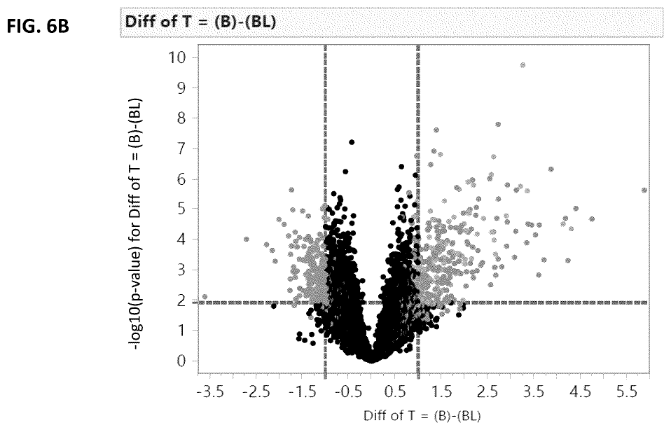

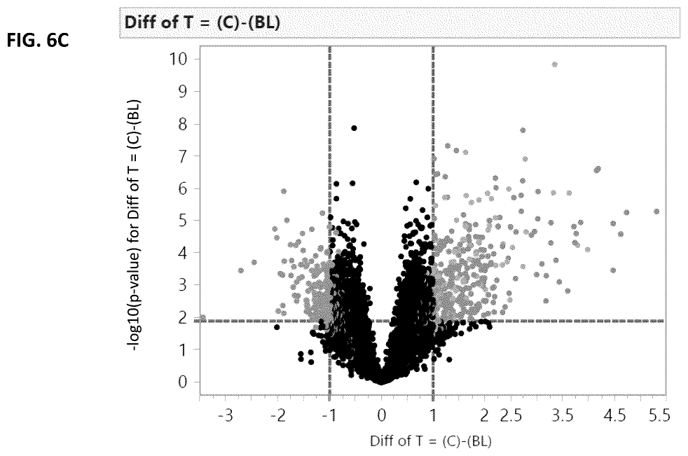

For each treatment group (A, B, C), gene expression levels were analyzed compared to the base line levels (BL), and differentially expressed genes (DEG) are presented in a Venn diagram (FIG. 5). The differentially expressed genes, are genes showing at least 2 folds change (FC>=2) compared to base line level. As demonstrated in FIG. 5, 31 genes are differentially expressed in group A, compared to base line levels (BL). Additionally, more than 500 genes are differentially expressed in groups B or C, compared to base line levels (BL). Notably, 376 DEGs are common between groups B and C, compared to base line levels (BL). 362 of the 376 DEGs are common only between groups B and C, and 14 DEGs are common for treatment groups A, B, and C, compared to base line levels (BL). These results demonstrated that cell growth in 3D systems affects the cells more than osteogenic induction (one or more osteogenic inducers, for 2 days). Furthermore, induction and reduction of DEGs compared to base line levels (BL), is more significant for groups B and C than group A (FIG. 6).

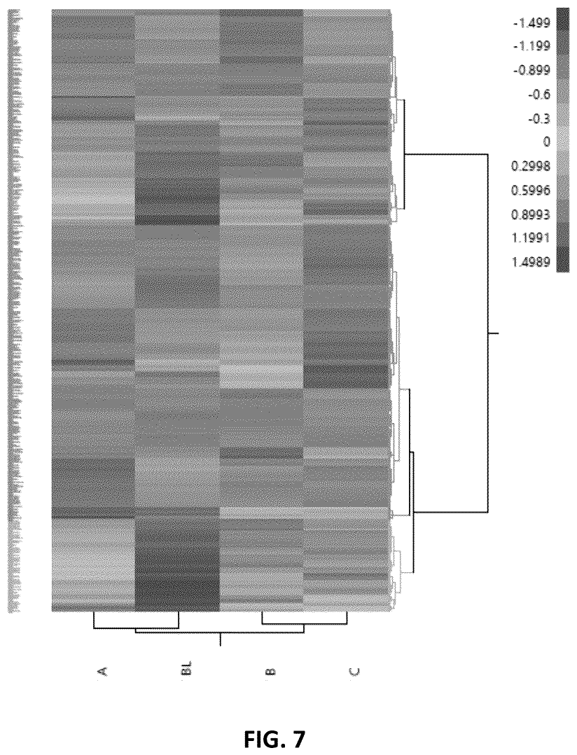

Moreover, the heat map shown in FIG. 7, demonstrates that treatments groups A and BL are similar and significantly different from treatment groups B and C, which are similar.

The microarray analysis demonstrates that when HATDCs are expanded in 3D, significant modifications in gene expression profile occurs. In contrast, when identical cells are grown in 2D conditions, with the same medium and osteogenic induction conditions (BMP2, 2d) the gene expression profile is very similar to the baseline control (2D, no BMP2 conditions) and is very different from the gene expression profile obtained at 3D conditions (groups B and C).

### Example 3

### Microarray Analysis Results

The microarray results were analyzed and DEGs were grouped into different clusters. Results demonstrated that only 14 DEGs are common for treatment groups which were subjected to osteogenic induction with one or more osteogenic inducers (groups A, B, and C), compared to base line levels (BL) (Table 12).

HATDCs, subjected to osteogenic induction for 48 hours, were found to exhibit modulation in expression levels of the genes ATOH8, CGB1, CMTM4, FOXO, ID1, ID2, ID3, NEBL, OSR1, PRRX2, SAMD11, SLC16A3, and SMAD9. Specifically, following osteogenic induction (groups A, B, and C) the expression level of the genes: ATOH8, CGB1, CMTM4, FOXO, ID1, ID2, ID3, NEBL, PRRX2, SAMD11, SLC16A3, and SMAD9 was induced, compared to HATDCs that were not subjected to osteogenic treatment (BL). The expression level of the gene OSR1, in HATDCs subjected to osteogenic induction (groups A, B, and C) was reduced, compared to HATDCs that were not subjected to osteogenic induction (BL).

TABLE 12
Common DEGs between treatment groups A, B, and C

| Gene | Entrez gene | | | |
|---|---|---|---|---|
| symbol | (http://www.ncbi.nlm.nih.gov/gene) | A-BL | B-BL | C-BL |
| ATOH8 | 84913 | 6.15 | 5.98 | 5.51 |
| CGB1 | 114335 | 2.53 | 2.52 | 3.06 |
| CMTM4 | 146223 | 3.13 | 3.17 | 2.99 |
| FOXO1 | 2308 | 2.23 | 4.23 | 4.14 |
| ID1 | 3397 | 11.87 | 10.26 | 12.35 |
| ID2 | 3398 | 3.51 | 5.33 | 5.63 |
| ID3 | 3399 | 8.17 | 9.58 | 10.16 |
| NEBL | 10529 | 2.58 | 2.78 | 3.24 |
| OSR1 | 130497 | −3.94 | −3.70 | −3.36 |
| PRRX2 | 51450 | 5.41 | 9.26 | 10.00 |
| SAMD11 | 148398 | 5.16 | 6.20 | 6.86 |
| SLC16A3 | 9123 | 2.49 | 2.49 | 3.34 |
| SMAD9 | 4093 | 2.07 | 2.80 | 3.09 |

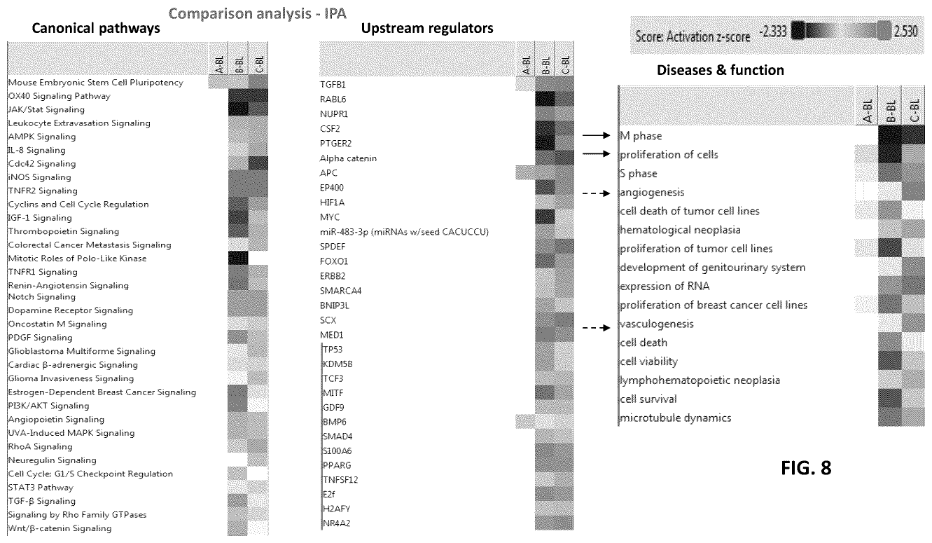

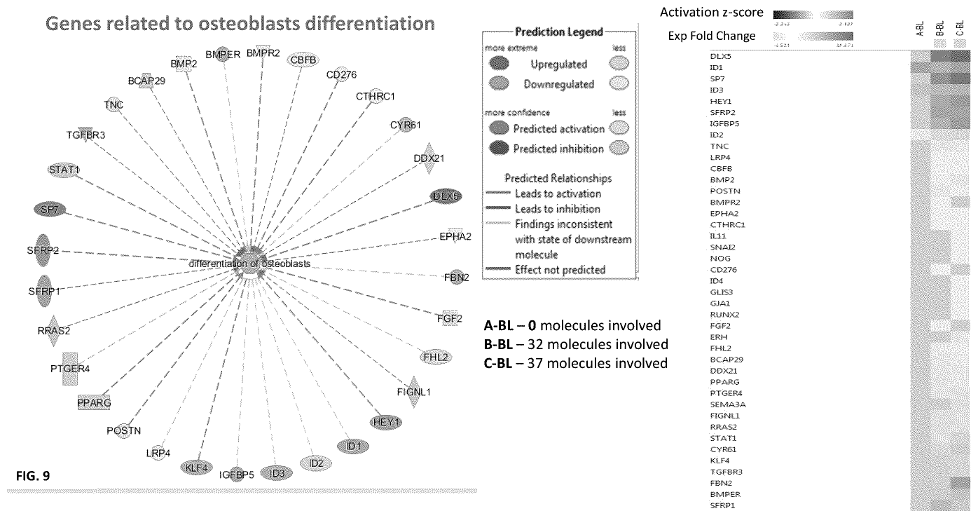

FIG. 8 demonstrates comparison of descriptors related to canonical pathways (left), upstream regulators (middle) and function analysis induced or reduced in the different tested growth conditions. The 3 treatment comparison was done by IPA analysis tool. Each descriptor shown here represents many related DEGs which induced or reduced compared to the baseline (BL) treatment. The overall effect of all related DEGs per descriptor is summarized and demonstrated in these heat maps. The results show that the two 3D growth conditions (B and C treatments) significantly affect the mentioned descriptors while 2D growth condition (treatment A) has minor effect relative to the baseline (BL).

### Stem Cells Markers

Expression level of pluripotent/multipotent stem cells markers including: CD13, CD73, CD90, and KLF4 is reduced in HATDCs grown in 3D systems (groups B and C) compared to control (BL) (Tables 1 and 1b). These results, indicate that HATDCs grown in 3D system undergo enhanced differentiation.

TABLE 1b
Mesenchymal stem cells related markers

| | | A-BL | B-BL | C-BL |
|---|---|---|---|---|
| Gene symbol | Gene full name | FC | FC | FC |
| ANPEP (CD13) | Alanyl (Membrane) | −1.36 | −2.4 | −2.06 |
| | Aminopeptidase | | | |
| NT5E (CD73) | 5′-Nucleotidase, Ecto | 1.18 | −1.7 | −1.42 |
| THY1 (CD90) | Thy-1 Cell Surface Antigen | −1.41 | −1.72 | −2.36 |
| KLF4 | Kruppel-Like Factor 4 (Gut) | −2.2 | −3.4 | −2.99 |

### Proliferation Differentiation and Apoptosis Markers

HATDCs grown in 3D systems exhibit reduced proliferation and enhanced differentiation. Microarray results demonstrate that, HATDCs grown in 3D systems exhibit increased expression of cell marker: AURKA, FOS, FGF2 (bFGF), BCL2L1, DDX21, RRAS2, STAT1, and ANXA2. In addition, HATDCs grown in 3D systems exhibit increased expression of cell marker including: SFRP2, ID1, ID2, ID3, MRAS, NOX4, NOTCH3, and RGCC (Tables 2 and 2b).

TABLE 2b
Proliferation markers reduced in 3D systems

| | | A-BL | B-BL | C-BL |
|---|---|---|---|---|
| Gene symbol | Gene full name | FC | FC | FC |
| AURKA | Aurora Kinase A | 1.18 | −2.4 | −1.95 |
| FOS | FBJ Murine Osteosarcoma Viral Oncogene | −7.6 | −12.13 | −10.8 |
| | Homolog | | | |
| FGF2 (bFGF) | Fibroblast Growth Factor 2 (Basic) | 1.4 | −1.6 | −1.5 |
| BCL2L1 | BCL2-Like 1 | 1.15 | −1.76 | −1.97 |
| DDX21 | DEAD (Asp-Glu-Ala-Asp) Box Helicase 21 | −1.14 | −2.04 | −1.9 |
| RRAS2 | Related RAS Viral (R-Ras) Oncogene Homolog 2 | −1 | −2.49 | −2.37 |
| STAT1 | Signal Transducer And Activator Of Transcription | −1.44 | −2.3 | −3.1 |
| | 1, 91 kDa | | | |
| ANXA2 | Annexin A2 | 1.39 | −3.2 | −2.7 |
| SFRP2 | Secreted Frizzled-Related Protein 2 | −1.52 | 12.15 | 12.10 |
| ID1 | Inhibitor Of DNA Binding 1, (2, 3,) Dominant | 11.87 | 10.26 | 12.35 |
| | Negative Helix-Loop-Helix Protein | | | |
| ID2 | Inhibitor Of DNA Binding 2, Dominant Negative | 3.5 | 5.3 | 5.6 |
| | Helix-Loop-Helix Protein | | | |
| ID3 | Inhibitor Of DNA Binding 3, Dominant Negative | 8.17 | 9.6 | 10.16 |
| | Helix-Loop-Helix Protein | | | |
| MRAS | Muscle RAS Oncogene Homolog | 1.06 | 2.31 | 2.17 |
| NOX4 | NADPH Oxidase 4 | 1 | 3.75 | 2.8 |
| NOTCH3 | Notch 3 | 1.2 | 5.84 | 6.6 |
| RGCC | Regulator of cell cycle | 1.35 | 3.44 | 4.34 |

### MHC I Proteins

Major Histocompatibility Complex (MHC) antigens are expressed almost in all differentiated cells. These proteins are involved in the presentation of foreign antigens to the immune system. MSCs are known to express low levels of MHC class I molecules.

MHC I genes are induced in HATDCs grown in 3D systems relative to HATDCs grown in 2D systems indicating for enhanced differentiation of the cells in 3D systems (Tables 3, 3b).

TABLE 3b
MHC I genes

| | | A-BL | B-BL | C-BL |
|---|---|---|---|---|
| Gene symbol | Gene full name | FC | FC | FC |
| HLA-A | Major Histocompatibility Complex, | −1.19 | 2.29 | 2.46 |
| | Class I, A | | | |
| HLA-B | Major Histocompatibility Complex, | −1.68 | 4.10 | 4.56 |
| | Class I, B | | | |
| HLA-DMA | Major Histocompatibility Complex, | 1.02 | 1.63 | 1.82 |
| | Class II, DM Alpha | | | |
| HLA-F | Major Histocompatibility Complex, | −1.41 | 3.47 | 3.80 |
| | Class I, F | | | |
| HLA-G | Major Histocompatibility Complex, | −1.13 | 2.08 | 2.47 |
| | Class I, G | | | |
| HLA-H | Major Histocompatibility Complex, | −1.30 | 3.82 | 3.86 |
| | Class I, H | | | |

### Adipocyte Markers

Gene markers of matured adipocytes (e.g.: PPARG) were reduced since the cells were already committed to bone differentiation (Tables 4, 4b). However, early adipocytes markers are induced in 3D (e.g.: DLK1, SOX9) (Tables 4, 4b). These results may suggest that under suitable growth conditions, the cells grown in 3D systems have the potential to differentiate into adipocytes as well as to bone.

TABLE 4b
Adipocyte markers

| Gene | | A-BL | B-BL | C-BL |
|---|---|---|---|---|
| symbol | Gene full name | FC | FC | FC |
| PPARG | Peroxisome Proliferator-Activated | −1.3 | −2.12 | −1.89 |
| | Receptor Gamma | | | |
| DLK1 | Delta-Like 1 Homolog (Drosophila) | 1 | 1.8 | 1.9 |
| ACSL1 | Acyl-CoA Synthetase Long-Chain | −1.33 | −2.06 | −1.86 |
| | Family Member 1 | | | |
| AEBP1 | AE Binding Protein 1 | −1.78 | 2.90 | 2.79 |
| Sox9 | SRY (Sex Determining Region Y)- | 1.01 | 2.73 | 2.53 |
| | Box 9 | | | |

### Osteoblasts Markers

Gene markers of this cluster are critical for osteoblasts differentiation (e.g., endogenous BMP2, SP7, and ALP) which is enhanced in the 3D conditions relative to the 2D.

Results demonstrated induction of markers such as BMP2, SP7 and ALP, these results are further supported by qRT-PCR results (FIGS. 1A-C, 2A-C, and 3A-C). Other important markers of bone differentiation obtained in 3D are: POSTN, FGFR3 and DLX5. Induction of Msx1 and Msx2 indicate for a development process and also for mechanism of bone differentiation occur in Neural crest cells (Tables 5, 5b).

Results obtained following IPA (see, Ingenuity Pathway Analysis-http://www.ingenuity.com/products/ipa) analysis demonstrate that 3D growth conditions (groups B, and C) results in more than 30 DEGs which are involved in osteoblasts differentiation, while group A (grown in a 2D system and subjected to osteogenic induction) did not result in DEGs which are involved in this pathway.

TABLE 5b
Osteoblast markers

| | | A-BL | B-BL | C-BL |
|---|---|---|---|---|
| Gene symbol | Gene full name | FC | FC | FC |
| BMP2 | Bone Morphogenetic Protein 2 | −1.04 | 3.05 | 3.80 |
| BMPR2 | Bone Morphogenetic Protein Receptor, Type II | 1.34 | 2.20 | 2.03 |
| SP7 | Sp7 Transcription Factor | 1.20 | 14.60 | 17.79 |
| AlPL | Alkaline Phosphatase, Liver/Bone/Kidney | −4.91 | 1.32 | 2.50 |
| POSTN | Osteoblast Specific Factor 2 | −1.42 | 2.85 | 2.59 |
| FGFR3 | Fibroblast Growth Factor Receptor 3 | 1.90 | 17.54 | 13.82 |
| Msx1 (Hox7) | Msh Homeobox 1 | −1.01 | 1.91 | 1.78 |
| Msx2 (Hox8) | Msh Homeobox 2 | −1.10 | 2.51 | 2.09 |
| DLX5 | Distal-Less Homeobox 5 | 1.81 | 16.96 | 19.37 |
| KAZALD1 | Kazal-Type Serine Peptidase Inhibitor Domain 1 | 1.41 | 2.07 | 1.98 |
| CA12 | Carbonic Anhydrase XII | −1.46 | 2.94 | 5.06 |
| BMPER | BMP Binding Endothelial Regulator | −1.54 | −3.64 | −4.28 |
| FBN2 | Fibrillin 2 | 1.12 | −3.4 | −6.5 |

Osteochondral Progenitors and/or Hypertrophic Chondrocytes Gene Markers

Osteoclasts markers are specific to cartilage development and to chondrocytes, osteochondral progenitors and hypertrophic chondrocytes. These gene markers indicate that the bone differentiation mechanism is involved endochondral ossification (Tables 6, 6b). Specific markers are: COL10A1, MMP13 and COMP.

TABLE 6b
osteochondral progenitors and/or hypertrophic chondrocytes
gene markers

| Gene | | A-BL | B-BL | C-BL |
|---|---|---|---|---|
| symbol | Gene full name | FC | FC | FC |
| Sox9 | SRY (Sex Determining Region Y)- | 1.01 | 2.73 | 2.53 |
| | Box 9 | | | |
| MGP | Matrix Gla Protein | 1.35 | 13.16 | 10.8 |
| COL10A1 | Collagen, Type X, Alpha 1 | 1.94 | 11.27 | 11.32 |
| COL9A2 | Collagen, Type IX, Alpha 2 | 1.11 | 2.42 | 2.05 |
| MMP13 | Matrix Metallopeptidase 13 | −1.00 | 4.58 | 4.00 |
| GSN | Gelsolin | 1.01 | 1.88 | 1.76 |
| CBFB | Core-Binding Factor, Beta Subunit | 1.26 | 2.42 | 2.58 |
| BAPX1 | NK3 Homeobox 2 | 1.11 | 1.76 | 1.62 |
| (NKX3-2) | | | | |
| RUNX1 | Runt-Related Transcription Factor 1 | −1.13 | 1.83 | 1.48 |
| RUNX2 | Runt-Related Transcription Factor 2 | −1.48 | 1.24 | 1.71 |
| COMP | Cartilage Oligomeric Matrix Protein | 1.79 | 58.91 | 39.70 |

### ECM Markers and Structural Proteins

The gene markers of ECM (Tables 7, 7b) and structural proteins (Tables 8, 8b) indicate for enhanced differentiation of the cells in 3D. Moreover, several ECM proteins are generated from hypertrophic chondrocytes during endochondral ossification process. Main ECM markers are: TNC and DPT genes.

TABLE 7b
ECM markers

| | | A-BL | B-BL | C-BL |
|---|---|---|---|---|
| Gene symbol | Gene full name | FC | FC | FC |
| BGN | Biglycan | −1.6 | 3.06 | 3.38 |
| LAMA4 | Laminin, Alpha 4 | −3 | 1.07 | 1.6 |
| LAMA2 | Laminin, Alpha 2 | −1.2 | 2.86 | 2.79 |
| LTBP3 | Latent Transforming Growth | 1.1 | 1.83 | 1.72 |
| | Factor Beta Binding Protein 3 | | | |
| DPT | Dermatopontin | −1.72 | 12.26 | 13.55 |
| EFEMP2 | EGF Containing Fibulin-Like | 1.41 | 2.52 | 2.63 |
| | Extracellular Matrix Protein 2 | | | |
| PLOD1 | Procollagen-Lysine, 2-Oxoglutarate | −1.05 | 1.63 | 1.67 |
| | 5-Dioxygenase 1 | | | |
| TNC | Tenascin C | 1.75 | 3.87 | 3.57 |
| DCN | Decorin | −1.17 | 2.1 | 3.05 |
| FBLN2 | Fibulin 2 | 1.3 | 5.85 | 6.82 |
| NDNF | Neuron-Derived Neurotrophic Factor | −1.15 | 1.85 | 3.7 |
| SULF1 | Sulfatase 1 | 1.3 | 18.14 | 22.2 |

TABLE 8b
Genes encoding structural proteins

| | | A-BL | B-BL | C-BL |
|---|---|---|---|---|
| Gene symbol | Gene full name | FC | FC | FC |
| MMP14 | Matrix Metallopeptidase 14 | −1.09 | 1.79 | 1.48 |
| MMP2 | Matrix Metallopeptidase 2 | −1.34 | 2.05 | 1.96 |
| MMP23B | Matrix Metallopeptidase 23B | −1.03 | 2.2 | 1.69 |
| MMP3 | Matrix Metallopeptidase3 | −1.27 | 18.8 | 22.2 |
| MMP7 | Matrix Metallopeptidase 7 | 1.07 | 4.5 | 4.3 |
| COL16A1 | Collagen, Type XVI, Alpha 1 | 1.07 | 2.1 | 2.35 |
| COL24A1 | Collagen, Type XXIV, Alpha 1 | 1.04 | 3.39 | 3.13 |
| COL6A2 | Collagen, Type VI, Alpha 2 | −1.36 | 3.12 | 3.93 |
| COL7A1 | Collagen, Type VII Alpha 1 | −1.3 | 2 | 2.07 |
| COL8A2 | Collagen, Type VIII, Alpha 2 | −1.09 | 2.6 | 2.3 |
| ADAMTS2 | ADAM Metallopeptidase With | 1.05 | 1.98 | 2.2 |
| | Thrombospondin Type 1 Motif, 2 | | | |
| PCOLCE | Procollagen C-Endopeptidase | −1.2 | 2.14 | 2.45 |
| | Enhancer | | | |

### Angiogenic and Vascularogenic Genes

The angiogenic and vasclorogenic gene markers contribute to angiogenesis and vascularogenesis processes. Some are growth factors or cytokines, such as: PGF and IL8. Others are specific mediators of blood vessels formation such as: ANG, ANGPT2 and ANGPTL2. Typically, during extensive osteogenesis, mainly via endochondral ossification process, angiogenesis is enhanced.

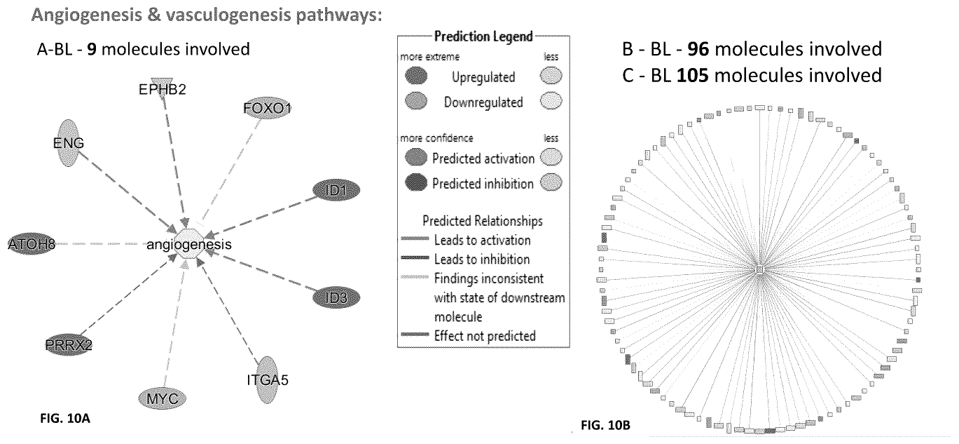

Results demonstrate that many angiogenic factors are induced in 3D compared with 2D growth conditions (Tables 9, 9b). Moreover, results from IPA analysis demonstrate that angiogenesis and vascularogenesis pathways are significantly induced (FIG. 8, right, marked by dashed arrows), and involved 96 (group B) and 105 (group C) of related DEGs (FIG. 10B). In contrast, in 2D growth conditions (group A) these processes are not induced (FIG. 8, right, marked by dashed arrows and FIG. 10A).

TABLE 9b
Expression of vascular markers

| | | A-BL | B-BL | C-BL |
|---|---|---|---|---|
| Gene symbol | Gene full name | FC | FC | FC |
| TBX2 | T-Box 2 | 1.56 | 2.45 | 1.99 |
| TBX3 | T-Box 3 | 1.05 | 2.54 | 3.00 |
| ANG | Angiogenin, Ribonuclease, RNase A Family, 5 | 1.02 | 1.67 | 2.52 |
| ANGPT2 | Angiopoietin 2 | 1.25 | 3.26 | 2.58 |
| ANGPTL2 | Angiopoietin-Like 2 | −1.93 | 12.08 | 9.05 |
| TRO | Trophinin | −1.01 | 2.07 | 2.11 |
| EDNRA | Endothelin Receptor Type A | −1.06 | 4.95 | 4.24 |
| EPHA2 | EPH Receptor A2 | 1.13 | 2.10 | 2.28 |
| F2R | Coagulation Factor II (Thrombin) Receptor | 1.28 | 1.75 | 1.73 |
| PGF | Placental Growth Factor | 1.04 | 2.07 | 2.47 |
| CTHRC1 | Collagen Triple Helix Repeat Containing 1 | 1.16 | 2.13 | 2.02 |
| PTGDS | Prostaglandin D2 Synthase 21 kDa (Brain) | −1.21 | 5.24 | 2.88 |
| AEBP1 | AE Binding Protein 1 | −1.78 | 2.90 | 2.79 |
| IL8 (Cxcl8) | Chemokine (C—X—C Motif) Ligand 8 | −1.20 | 2.63 | 2.17 |
| IL11 | Interleukin 11 | 1.02 | 1.62 | 2.09 |
| HEY1 | Hes-Related Family BHLH Transcription Factor | 1.69 | 11.58 | 13.3 |
| | With YRPW Motif 1 | | | |
| ECM1 | Extracellular Matrix Protein 1 | −1.13 | 1.9 | 1.65 |
| MFGE8 | Milk Fat Globule-EGF Factor 8 Protein | 1.05 | 2.78 | 2.41 |
| SRPX2 | Sushi-Repeat Containing Protein, X-Linked 2 | 1.52 | 3.42 | 2.68 |
| UNC5B | Unc-5 Homolog B (C. Elegans) | 1.4 | 4.08 | 3.55 |

### Expression of Upstream Regulators

Results demonstrate that upstream regulators are induced in 3D compared with 2D growth conditions (Tables 10, 10b).

TABLE 10b
Expression of upstream regulators

| Gene | | A-BL | B-BL | C-BL |
|---|---|---|---|---|
| symbol | Gene full name | FC | FC | FC |
| TGFB3 | Transforming Growth Factor, | −1.81 | 2.66 | 3.30 |
| | Beta 3 | | | |
| BAMBI | BMP And Activin Membrane- | 1.45 | 10.16 | 8.57 |
| | Bound Inhibitor | | | |
| IGFBP2 | Insulin-Like Growth Factor | −1.04 | 3.09 | 1.76 |
| | Binding Protein 2, 36 kDa | | | |
| IGFBP5 | Insulin-Like Growth Factor | −1.48 | 6.49 | 8.11 |
| | Binding Protein 5 | | | |

### Additional DEGs Having Significant Modulation in Expression Levels

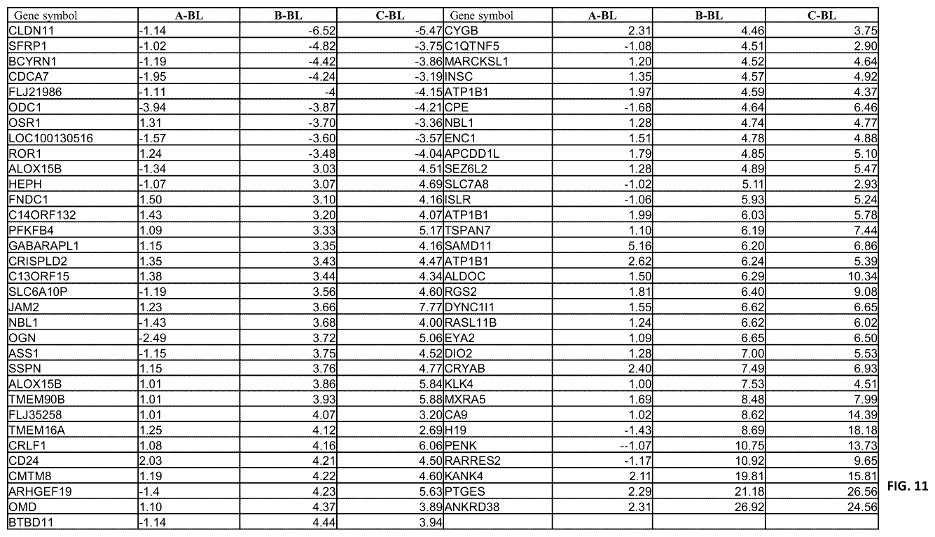

Table 11b demonstrates DEGs having at least 3 folds change (see FIG. 11).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

What is claimed is:

1. A method for identifying a cell population suitable for transplantation to a subject in need thereof, the method comprising determining the expression levels of a plurality of genes in a cell population, wherein differences in expression levels of a plurality of genes selected from the genes selected from at least two tables selected from tables 1-11, compared to a control expression levels, indicates that said cell population is suitable for transplantation.

2. The method of claim 1, wherein the cell population is derived from human adipose tissue derived cells (HATDCs).

3. The method of claim 1, wherein the cell population the subjected to osteogenic differentiation.

4. The method of claim 3, wherein said osteogenic differentiation is induced by an osteogenic inducer selected from the group consisting of: bone morphogenic protein (BMP)-2, BMP-3, BMP-4, BMP-5, BMP-6 and BMP-7.

5. The method of claim 1, wherein the cell population is grown in a three dimensional culture.

6. The method of claim 1, wherein said grown is in contact with a mineral particle.

7. The method of claim 6, wherein said mineral particle is selected from the group consisting of: coral mineral particle, cancellous bone and cortical bone.

8. The method of claim 1, wherein the control expression levels corresponding to a second cell population derived from cells grown in a two dimensional culture and subjected to osteogenic induction.

9. The method of claim 1, wherein said differences in expression levels are, independently for each gene, selected from up-regulation, and down-regulation.

10. The method of claim 1, wherein said plurality of genes is selected from one or more genes of each one of tables 1-11.

11. The method of claim 1, wherein said plurality of genes is selected from the genes listed in a table selected from tables 1-11.

12. The method of claim 1, wherein said plurality of genes comprises at least 50% of the genes listed in tables 1-11.

13. The method of claim 1, wherein the plurality of genes comprises MARCKSL1 and one or more additional genes selected from at least two tables selected from tables 1-11.

14. The method of claim 1, wherein said differences comprises up-regulation of MARCKSL1 and at least one difference selected from: up-regulation of a plurality of genes selected from one or more genes selected from SFRP2, MRAS, NOX4, NOTCH3, and RGCC of Table 2; one or more genes selected from HLA-A, HLA-B, HLA-DMA, HLA-F, HLA-G, and HLA-H of Table 3; one or more genes selected from DLK1, AEBP1, and Sox9 of Table 4; one or more genes selected from BMP2, BMPR2, SP7, ALP, POSTN, FGFR3, Msx1, Msx2, DLX5, KAZALD1, and CA12 of Table 5; one or more genes selected from Sox9, MGP, COL10A1, COL9A2, MMP13, GSN, CBFB, BAPX1, RUNX1, RUNX2, and COMP of Table 6; one or more genes selected from BGN, LAMA4, LAMA2, LTBP3, DPT, EFEMP2, PLOD1, TNC, DCN, FBLN2, NDNF, and SULF1 of Table 7; one or more genes selected MMP14, MMP2, MMP23B, MMP3, MMP7, COL16A1, COL24A1, COL6A2, COL7A1, COL8A2, ADAMTS2 of Table 8; one or more genes selected TBX2, TBX3, ANG, ANGPT2, ANGPTL2, TRO, EDNRA, EPHA2, F2R, PGF, CTHRC1, PTGDS, AEBP1, IL8 (Cxcl8), IL11, HEY1, ECM1, MFGE8, and SRPX2, and UNC5B of Table 9; one or more genes selected from TGFB3, BAMBI, IGFBP2, and IGFBP5 of Table 10; one or more genes selected from ALOX15B, HEPH, FNDC1, C14ORF132, PFKFB4, GABARAPL1, CRISPLD2, C13ORF15, SLC6A10P, JAM2, NBL1, OGN, ASS1, SSPN, ALOX15B, TMEM90B, FLJ35258, TMEM16A, CRLF1, CD24, CMTM8, ARHGEF19, OMD, BTBD11CYGB, C1QTNF5, INSC, ATPIB1, CPE, NBL1, ENC1, APCDD1L, SEZ6L2, SLC7A8, ISLR, ATPIB1, TSPAN7, SAMD11, ATPIB1, ALDOC, RGS2, DYNC1I1, RASL11B, EYA2, DIO2, CRYAB, KLK4, MXRA5, CA9, H19, PENK, RARRES2, KANK4, PTGES, and ANKRD38 compared to said control; and down-regulation of a plurality of genes selected from one or more genes selected from ANPEP, NT5E, THY1, and KLF4 of Table 1; one or more genes selected from AURKA, FOS, FGF2, BCL2L1, DDX21, RRAS2, STAT1, and ANXA2 of Table 2; one or more genes selected from PPARG, and ACSL1 of Table 4; one or more genes selected from BMPER, and FBN2 of Table 5, one or more genes selected from CLDN1, SFRP1, BCYRN, CDCA7, FLJ21986, ODC1, OSR1, LOC100130516, and ROR1 of Table 11, compared to said control.

15. The method of claim 1, wherein said determining step comprises the step of obtaining nucleic acid molecules from said cell population.

16. The method of claim 15, wherein said nucleic acids molecules are selected from mRNA molecules, DNA molecules and cDNA molecules.

17. The method of claim 1, wherein said determining further comprises the step of hybridizing said nucleic acid molecules with a plurality of ligands each ligand capable of specifically complexing with, binding to, hybridizing to, or quantitatively detecting or identifying a single gene selected from the genes listed in Tables 1-11.
